(19) United States
(12) Patent Application Publication
(10) Pub. No.: US 2021/0011006 A1
(43) Pub. Date: Jan. 14, 2021

(54) ENGINEERED IMMUNE CELLS AS DIAGNOSTIC PROBES OF DISEASE

(71) Applicant: The Board of Trustees of the Leland Stanford Junior University, Stanford, CA (US)

(72) Inventors: Amin AALIPOUR, Stanford, CA (US); Sanjiv S. GAMBHIR, Stanford, CA (US)

(21) Appl. No.: 16/969,929

(22) PCT Filed: Feb. 27, 2019
(86) PCT No.: PCT/US2019/019787

§ 371 Date: Aug. 13, 2020

Related U.S. Application Data
(60) Provisional application No. 62/635,664, filed on Feb. 27, 2018.
(60) Provisional application No. 62/794,011, filed on Jan. 18, 2019.

(51) Int. Cl. G01N 33/50; C12N 5/0786; C12N 15/85; G01N 33/574

(52) U.S. Cl. CPC G01N 33/5091; C12N 5/0645; C12N 2830/002; G01N 33/57488; C12N 2510/00; C12N 15/85

(57) ABSTRACT

Embodiments of genetically engineered immune cells are described herein which provide a new class of cell-based in vivo sensors useful for ultrasensitive disease detection based on the ability of immune cells to migrate to a site of pathology. The cell-based sensors provide an approach to early cancer detection and allow the use of the engineered immune cells in monitoring of diverse disease states including, but not limited to, cancer.

### CROSS-REFERENCE TO RELATED APPLICATION

This application claims priority to US Provisional Application No. 62/635,664, entitled “ENGINEERED IMMUNE CELLS AS DIAGNOSTIC PROBES OF DISEASE” filed on Feb. 27, 2018, and to US Provisional Application No. 62/794,011, entitled “ENGINEERED IMMUNE CELLS AS DIAGNOSTIC PROBES OF DISEASE” filed on Jan. 18, 2019, the entireties of which are herein incorporated by reference.

### SEQUENCE LISTING

This application contains a sequence listing filed in electronic form as an ASCII.txt file entitled “2219072430_ST25” created on Feb. 13, 2019. The content of the sequence listing is incorporated herein in its entirety.

### TECHNICAL FIELD

The present disclosure is generally related to tumor- or other disease-specific probes comprising genetically engineered immune cells. The present disclosure is also generally related to methods of making and using the probes.

### BACKGROUND

Early detection of primary disease and recurrence are promising avenues towards significantly reducing the global cancer burden. To date, most early detection efforts have relied on detection of endogenous biomarkers characteristic of a disease state. In cancer, for example, biomarkers including proteins, circulating tumor cells (CTCs), cell-free circulating tumor DNA (ctDNA), cancer-derived exosomes, tumor educated platelets, and microRNAs have been the subject of much investigation. Endogenous biomarkers remain at the forefront of early disease detection efforts, but many lack the sensitivities and specificities necessary to influence disease management.

### SUMMARY

The present disclosure encompasses embodiments of a novel engineered (genetically modified) macrophage cells, which may or may not have been originally isolated from a patient. The genetic modification is the introduction into the cells of a gene expression cassette encoding a detectable polypeptide, or a nucleic acid such as a miRNA, and under the expression control of a gene promoter/enhancer inducible by a tumor- or disease-specific metabolic factor.

Accordingly, one aspect of the disclosure encompasses embodiments of a genetically modified immune cell comprising a heterologous nucleic acid configured to express a detectable agent in response to a metabolic or molecular expression change induced by a pathological condition in an animal or human subject receiving the genetically modified immune cell.

In some embodiments of this aspect of the disclosure, the genetically modified immune cell can be a monocyte, a macrophage, a T-cell, a B-cell, a natural killer (NK) cell, myeloid cell, stem cell, or a dendritic cell.

In some embodiments of this aspect of the disclosure, the heterologous nucleic acid can comprise at least one gene expression cassette comprising a gene expression regulatory region operably linked to a nucleic acid sequence encoding a detectable agent, and wherein the gene expression regulatory region can be responsive to a pathology-specific metabolic change in the genetically modified immune cell to induce expression of the detectable agent.

In some embodiments of this aspect of the disclosure, the heterologous nucleic acid can be a nucleic acid vector.

In some embodiments of this aspect of the disclosure, the heterologous nucleic acid can be a plasmid.

In some embodiments of this aspect of the disclosure, the gene expression regulatory region can comprise a gene promoter region.

In some embodiments of this aspect of the disclosure, the gene expression regulatory region can further comprise a gene-specific enhancer.

In some embodiments of this aspect of the disclosure, the gene promoter can be an ARG1 promoter, an AKT1 promoter, a versican promoter, a MIF promoter, a Ym1 promoter, a CD206 promoter, a FIZZ1 promoter, a DC-SIGN promoter, a CD209 promoter, a MGL-1 promoter, a Dectin -1 promoter, a CD23 promoter, a galectin-3 promoter, a Mer tyrosine kinase promoter, an AXL receptor protein promoter, a GAS-6 promoter, a NOS-2 promoter, a CD68 promoter, a CD86 promoter, a CCL18 promoter, a CD163 promoter, a MMR/CD206 promoter, a CD200R promoter, a TGM2 promoter, a DecoyR promoter, an IL-1 R II promoter, an IL-10 promoter, a TGF-beta promoter, an IL-1ra promoter, a CCL17 promoter, a CCL2 promoter, or a CCL24 promoter.

In some embodiments of this aspect of the disclosure, the detectable agent can be a detectable polypeptide or a secretable nucleic acid.

In some embodiments of this aspect of the disclosure, the detectable polypeptide can be a contrast agent, a binding agent complementary to a reporter gene, an enzyme producing a detectable molecule, a photoacoustic reporter, a bioluminescent reporter, an autofluorescent reporter, a chemiluminescent reporter, a luminescent reporter, or a colorimetric reporter, an agent that can be detected by non-invasive imaging or a transporter driving accumulation of a detectable molecule.

In some embodiments of this aspect of the disclosure, the detectable agent can be a secretable nucleic acid.

In some embodiments of this aspect of the disclosure, the secretable nucleic acid can be a structured RNA or a synthetic miRNA detectable by RT-QPCR, QPCR, hybridization, sequencing, or mass spectroscopy.

In some embodiments of this aspect of the disclosure, the detectable polypeptide can be Gaussia luciferase (Gluc).

In some embodiments of this aspect of the disclosure, the detectable polypeptide can be ferritin.

In some embodiments of this aspect of the disclosure, the detectable polypeptide can be HSV1-thymidine kinase (HSV1-tk).

In some embodiments of this aspect of the disclosure, the detectable polypeptide can be a D8ORA mutant of the dopamine D2 receptor.

In some embodiments of this aspect of the disclosure, the detectable polypeptide can be a human sodium iodide symporter (hNIS).

In some embodiments of this aspect of the disclosure, the heterologous nucleic acid can have at least 80% identity to the nucleotide sequence as shown in SEQ ID NO: 1.

In some embodiments of this aspect of the disclosure, the gene expression regulatory region can be responsive to a tumor-specific metabolic change in the genetically modified immune cell to induce expression of the detectable agent.

In some embodiments of this aspect of the disclosure, the tumor-specific metabolic change in the genetically modified immune cell is induced by a cancer selected from the group consisting of: bladder cancer, breast cancer, colorectal cancer, endometrial cancer, head and neck cancer, lung cancer, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, liver cancer, medulloblastoma, neuroblastoma, brain tumor, osteosarcoma, malignant fibrous histiocytoma of bone, retinoblastoma, rhabdomyosarcoma, sarcoma, supratentorial primitive neuroectodermal tumor, pineal tumors, visual pathway and hypothalamic glioma, Wilms' tumor, esophageal cancer, hairy cell leukemia, kidney cancer, oral cancer, pancreatic cancer, skin cancer and small-cell lung cancer.

In some embodiments of the disclosure, the pathology-specific metabolic change in the genetically modified immune cell can be from an inflammation.

In some embodiments, the heterologous nucleic acid can comprise a plurality of different gene expression regulatory regions wherein each regulatory region is operably linked to a plurality of nucleic acid sequence encoding a multiple types of detectable agent, and wherein the gene expression regulatory region is responsive to a pathology-specific metabolic change to induce expression of the detectable agent, of which the levels of each detectable agent are indicative of a different condition of the subject.

Another aspect of the disclosure encompasses embodiments of a method of generating a genetically modified immune cell comprising the steps of:

(a) isolating from a subject a population of pathology-responsive immune cells; and

(b) transforming a pathology-responsive immune cell of the isolated population of pathology-responsive immune cells isolated in (a) with a heterologous nucleic acid to yield the genetically modified immune cell, wherein the heterologous nucleic acid encodes a detectable agent, and wherein the genetically modified immune cell is configured to express the detectable agent in response to a metabolic change induced by a pathological condition in an animal or human subject receiving the genetically modified immune cell.

In some embodiments of this aspect of the disclosure, the pathology can be a tumor.

In some embodiments of this aspect of the disclosure, the tumor-responsive immune cells are macrophages.

Yet another aspect of the disclosure encompasses embodiments of a method of detecting a pathological condition in an animal or human subject comprising the steps of: administering to an animal or human subject a pharmaceutically acceptable composition comprising a population of genetically-modified immune cells according to the disclosure; obtaining a biofluid sample from the animal or human subject; determining whether the biofluid sample comprises a secretable detectable agent expressed by the genetically-modified immune cells in contact with or in the proximity of a pathological condition of the animal or human patient.

In some embodiments of this aspect of the disclosure, the biofluid can be blood.

In some embodiments of this aspect of the disclosure, the presence of the secretable detectable agent indicates that the animal or human has a pathological condition inducing phenotypic change in the genetically-modified immune cells in contact with the pathological condition.

In some embodiments, the genetically modified-responsive immune cells are tumor-responsive macrophages.

In some embodiments of this aspect of the disclosure, the pathological condition can be a cancer.

In some embodiments, the pathological condition is a tumor.

In some embodiments of this aspect of the disclosure, the method can further comprise the step of detecting a signal from the detectable agent in pathology-responsive immune cells adjacent to or attaching to the pathological condition; generating an image of the detectable signal relative to the subject; and determining the position of the localized signal in the subject.

In some embodiments of this aspect of the disclosure, the biofluid is blood.

In some embodiments, the method is performed when an amount of the detectable agent is not secreted by the genetically-modified immune cells adjacent to or attaching to a pathological condition of the animal or human patient.

Still another aspect of the disclosure encompasses embodiments of a kit comprising an apparatus for bone marrow derived macrophage (BMDM) isolation; and an endotoxin-free preparation of a plasmid encoding a detectable agent operably linked to an ARG-1 promoter.

Another aspect of the disclosure encompasses embodiments of a method for identifying a pathological condition in a subject, comprising: (a) administering to the subject a genetically modified immune cell comprising a heterologous nucleic acid having a nucleic acid sequence that encodes a detectable agent, wherein the genetically modified immune cell expresses the detectable agent in response to a metabolic change induced by a pathological condition in the subject, and (b) detecting the detectable agent in the subject to identify the pathological condition.

In some embodiments of this aspect of the disclosure, when responsive to a tumor-specific metabolic change in the genetically modified immune cell, the gene expression regulatory region can induce expression of the detectable agent. In embodiments of this aspect of the disclosure, the tumor-specific metabolic change in the genetically modified immune cell can be induced by a cancer selected from the group consisting of: bladder cancer, breast cancer, colorectal cancer, endometrial cancer, head and neck cancer, lung cancer, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, liver cancer, medulloblastoma, neuroblastoma, brain tumor, osteosarcoma, malignant fibrous histiocytoma of bone, retinoblastoma, rhabdomyosarcoma, sarcoma, supratentorial primitive neuroectodermal tumor, pineal tumors, visual pathway and hypothalamic glioma, Wilms' tumor, esophageal cancer, hairy cell leukemia, kidney cancer, oral cancer, pancreatic cancer, skin cancer and small-cell lung cancer.

In some embodiments of this aspect of the disclosure, the pathology-specific metabolic change in the genetically modified immune cell is from an inflammation.

In some embodiments of the genetically modified immune cell the heterologous nucleic acid comprises a plurality of different gene expression regulatory regions wherein each regulatory region is operably linked to a plurality of nucleic acid sequence encoding a multiple types of detectable agent, and wherein the gene expression regulatory region is responsive to a pathology-specific metabolic change to induce expression of the detectable agent, of which the levels of each detectable agent are indicative of a different condition of the subject.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 schematically illustrates diagnostic adoptive cell transfer. Macrophages are genetically engineered to secrete a synthetic biomarker upon adopting a “tumor associated” metabolic profile. The engineered macrophages are injected intravenously in syngeneic hosts and allowed to home in on existing sites of pathology. A blood test (or test of other biofluid) can then be used to monitor for secretion of the biomarker from the engineered macrophage that indicates the presence of disease. This system can also provide spatial information of immune cell activation with use of an imageable synthetic biomarker. The term “Macs” denotes macrophages.

FIGS. 2A-2F illustrate that (a) M2 macrophages are highly represented across a range of human cancers and (b) that arginase-1 identifies M2 macrophages in vitro and in vivo mouse models of cancer.

FIG. 2A illustrates a Heat map depicting relative fractions of various immune cells across a range of human cancers.

FIG. 2B illustrates a series of bar graphs that show murine BMDMs exhibit concentration-dependent increases in ARG1 expression in response to IL-4, IL-13, and tumor conditioned media as measured with qPCR.

FIG. 2C illustrates a series of bar graphs that show RAW264.7 murine macrophages exhibit concentration-dependent increases in ARG1 expression in response to IL-4, IL-13, and tumor conditioned media as measured with qPCR.

FIG. 2D illustrates a series of bar graphs that show arginase activity assays demonstrating elevated ARG1 levels upon stimulation with IL-4, IL-13, and tumor conditioned media (TCM).

FIG. 2E illustrates a FACS plot and a bar graph that show endogenous macrophages and intravenously injected adoptively transferred (ACT) RAW264.7 macrophages were isolated from tumors and spleens of subcutaneous tumor bearing mice (left) and fold elevations in ARG1 expression found in tumor infiltrating macrophages compared to their splenic counterparts were quantified by qPCR (right). ARG1 levels from non-macrophage cells in the tumor (AO, all other) was also measured in bulk and quantified relative to splenic macrophages. * indicates statistical significance at p<0.05 and *** indicates statistical significance at p<0.001. Error bars depict standard error of the mean (s.e.m) of at least three biological replicates. BMDM, bone marrow-derived macrophages; TCM, tumor conditioned media; ACT, adoptive cell transfer.

FIG. 2F illustrates that endogenous macrophages and adoptively transferred (ACT) RAW264.7 macrophages were isolated by flow cytometry from tumors, spleens, lungs, and livers of subcutaneous tumor bearing or healthy mice (top left left) and fold elevations of M2 gene expression in different tissues relative to liver-resident (for endogenous) or liver-homing (for ACT) macrophages are shown (top right). * indicates statistical significance at p<0.05, ** indicates statistical significance at p<0.01, and *** indicates statistical significance at p<0.001. Error bars depict standard error of the mean (s.e.m). BMDM, bone marrow-derived macrophage; TCM, tumor conditioned media.

FIGS. 3A-3G illustrate that macrophages migrate towards tumors in vitro and in vivo

FIG. 3A illustrates transwell assays (diagrammed top) performed with tumor conditioned media as the chemoattractant revealed a concentration dependent increase in RAW264.7 macrophage migration by optical microscopy (right) with quantification of migrated cells per 10× field revealing greater than four-fold increases in migration (bottom). Macrophages are shown in red false-color. Scale bars measure 400 μm.

FIGS. 3B and 3C illustrate that tumor and macrophage signals strongly co-localize on radiance line traces as a function of distance from scruff across the right shoulder.

FIG. 3B is a series of digital photographs with superimposed false color showing VivoTrack 680 labeled macrophages demonstrate a time-dependent accumulation in a subcutaneous Fluc-expressing tumor from days 1 to 5 after intravenous injection as visualized with in vivo fluorescence microscopy. Scale bar measures 1 cm. Left and right radiance scales apply to the tumor and macrophage signals respectively.

FIG. 3C is a graph showing corresponding right shoulder radiance line trace to FIG. 3B of radiance versus distance showing tumor and macrophage signal strongly co-localize on radiance line traces as a function of distance from scruff across the right shoulder.

FIG. 3D is shows Flow cytometry (FACS plot) results of harvested tumor and spleens demonstrating that after the fifth day between 19-25% of resident macrophages in each site are from adoptive transfer (VivoTrack680 positive) suggesting tumor colonization and persistence of the macrophage sensor over time. FIG. 3E is a pie chart showing that after the fifth day between 19-25% of resident macrophages in each site are from adoptive transfer suggesting tumor colonization and persistence of the macrophage sensor over time.

FIGS. 3F and 3G illustrate that neutralizing doses of anti-CCL2 (n=4, p=0.0077) and anti-CSF1 (n=3, p=0.0049) antibody interferes with macrophage migration to subcutaneous tumors more so than their respective isotype control antibodies.

FIG. 3F illustrates digital photographs with superimposed false color showing that neutralizing doses of anti-CCL2 (n=4, p=0.0077) and anti-CSF1 (n=3, p=0.0049) antibody interferes with macrophage migration to subcutaneous tumors more so than their respective isotype control antibodies.

FIG. 3G illustrates a bar graph showing radiance values background subtracted. * indicates statistical significance at p<0.05, ** indicates statistical significance at p<0.01, and *** indicates statistical significance at p<0.001. Error bars depict s.e.m of at least three biological replicates. TCM, tumor conditioned media; Ab, antibody; AH, Armenian hamster.

FIGS. 4A-4I illustrate that macrophage sensors enable detection and visualization of small tumors in vivo.

FIG. 4A illustrates a pair of bar graphs showing that RAW264.7 macrophages engineered to express the pARG1-Gluc reporter exhibit a time and concentration dependent secretion of Gluc when stimulated with tumor conditioned media (left) and IL-4/IL-13 (right) as assayed from culture media.

FIG. 4B is a scatter plot showing RLU values assayed from plasma of 4T1 tumor bearing mice showed no elevations above healthy controls (n=7, AUC=0.657, 95% CI 0.335-0.979, p=0.372) in localized lung microtumors but significant elevations in disseminated disease (n=11, AUC=1.00, p=0.0018). RLU values are background subtracted to eliminate non-specific signal from healthy blood.

FIG. 4C is a series of digital photographs showing bioluminescent imaging (BLI) of activated macrophages and tumor dissemination revealing marked co-localization of macrophage sensor activation with sites of disease.

FIG. 4D is a scatter plot showing in a localized subcutaneous CT26 model, background subtracted plasma Gluc from activated macrophage sensor could reliably detect >50 mm³ tumors with an AUC=1.00 (n=6, p=0.0009) and 25-50 mm³ tumors with an AUC=0.849 (n=6, 95% CI 0.620-1.00, p=0.021).

FIG. 4E is a series of digital photographs of BLI imaging showing visible spatial overlap of activated macrophages (white circle) with right shoulder tumors. Scale bars measure 1 cm.

FIG. 4F is a graph showing right shoulder radiance line traces showing visible spatial overlap of activated macrophages (white circle) with right shoulder tumors. Scale bars measure 1 cm. Left and right radiance scales in FIGS. 4C and 4E apply to the activated macrophages and tumor signals respectively. * indicates statistical significance at p<0.05, ** indicates statistical significance at p<0.01, and *** indicates statistical significance at p<0.001. Error bars depict s.e.m of at least three biological replicates. TCM, tumor conditioned media; RLU, relative luminescence units; AUC, area under the curve.

FIG. 4G illustrates a fluorescent-activated cell sorting (FACS) trace showing bone marrow derived cells cultured in M-CSF exhibit increasing levels of monocyte/macrophage maturity marker F4/80 with a monocyte phenotype present at day 5 of culture.

FIG. 4H is a graph showing BMDMs electroporated with the pARG1-Gluc reporter exhibit time dependent secretion of Gluc with tumor conditioned media as assayed from culture media.

FIG. 4I illustrates a scatter plot showing background subtracted plasma Gluc from activated BMDM sensors shows significant elevation (n=4, p=0.0342) when localized subcutaneous CT26 tumors reach volumes of 60-75 mm3 (AUC=0.813, 95% CI 0.555-1.00, p=0.0894). Scale bars measure 1 cm. Left and right radiance scales in (C) and (E) apply to the activated macrophages and tumor signals respectively. * indicates statistical significance at p<0.05, ** indicates statistical significance at p<0.01, and *** indicates statistical significance at p<0.001. Error bars depict s.e.m. TCM, tumor conditioned media; RLU, relative luminescence units; AUC, area under the curve.

FIGS. 5A-5I illustrate macrophage sensors reflect immunological time frame in a model of inflammation and wound healing

FIG. 5A illustrates a bar graph showing BMDMs and RAW264.7 macrophages exhibit minimal elevations in ARG1 mRNA, as quantified by qPCR, upon exposure to classic pro-inflammatory cytokines IFNγ and TNFα as quantified by qPCR. BMDM ARG1 levels are similarly not affected by LPS.

FIG. 5B is a bar graph showing the engineered pARG1-Gluc expressing macrophage sensor sensors do not result in notably increased Gluc levels in culture media upon stimulation with the same pro-inflammatory cytokines.

FIG. 5C is a series of digital photographs showing H&E stains of hind leg muscle from days 0-10 post intramuscular injection of turpentine oil exhibit a classical timeline of acute inflammation with a primary neutrophilic (dark arrows) response followed by infiltration of macrophages (light arrows) in the later stages of inflammation resolution. Scale bars measure 50 μm.

FIG. 5D is a scatter plot showing background subtracted plasma Gluc levels 24 hours after intravenous macrophage sensor injection either on day 1 (n=6) or day 7 (n=8) of inflammation (left) revealed no elevation in the acute inflammatory phase (day 1) but significant elevation and macrophage activation in the resolution phase (day 7). This is also reflected by an undiscriminating AUC=0.643 (95% CI 0.332-0.953, p=0.371) during acute inflammation but a robust AUC=0.929 (95% CI 0.783-1.00, p=0.006) during the wound healing phase.

FIG. 5E is a series of digital bioluminescent imaging (BLI) images of intracellular Gluc from activated macrophages revealing comparable signal from background, non-inflamed mice injected with sensor (control), and acutely-inflamed mice injected with sensor (Acute Inf.). This contrasts with BLI images when macrophage sensor is injected during the resolution phase on day 7 wherein localized activation of macrophages at the site of wound healing (black circle) is clearly visible. Scale bar measure 1 cm. * indicates statistical significance at p<0.05 and *** indicates statistical significance at p<0.001. Error bars depict s.e.m of at least three biological replicates. RLU, relative luminescence units; AUC, area under the curve.

FIG. 5F is a series of digital photographs showing H&E stained micrographs of lungs following intranasal inoculation with LPS exhibit a similar timeline of acute inflammation with a neutrophilic infiltrate (green arrows) present at 7 hours followed by gradual replacement with macrophages (yellow arrows) as the wound healing process progresses. Wound healing peaks at 48 hours after LPS inoculation and by 72 hours there is some restoration of healthy lung architecture. Scale bars measure 50 μm.

FIG. 5G is a scatter plot showing plasma Gluc measurements of mice injected with BMDM sensor reflect the acute inflammation and wound healing kinetics peaking at 48 hours with an AUC=0.975 (95% CI 0.900-1.00, n=5, p=0.0054).

FIG. 5H and 5I illustrate a scatter plot (FIG. 5H) and a series of digital BLI images (FIG. 5I) showing the BMDM sensor can robustly discriminate metastatic 4T1 tumors both in the absence (AUC=0.975, 95% CI 0.900-1.00, n=5, p=0.0054) and presence (AUC=1.00, 95% CI 1.00-1.00, n=4, p=0.0066) of LPS-induced acute inflammation via plasma Gluc measurements (FIG. 5H) as well as (FIG. 5I) via BLI of activated macrophages * indicates statistical significance at p<0.05, ** indicates statistical significance at p<0.01, and *** indicates statistical significance at p<0.001. Error bars depict s.e.m. LPS, lipopolysaccharide; RLU, relative luminescence units; AUC, area under the curve.

FIGS. 6A-6F illustrate that macrophage sensors outperform a clinically used biomarker of cancer recurrence.

FIG. 6A is a graph showing that subcutaneously implanted LS174T tumors exhibit exponential growth in nu/nu mice (n=12).

FIG. 6B is a graph showing increasing levels of plasma CEA detected by enzyme-linked immunosorbent assay (ELISA).

FIG. 6C is a scatter plot showing that on day one of plasma sampling, the background subtracted plasma Gluc measurements from a macrophage sensor (left) were better able to discriminate tumor bearing (n=7) and healthy mice (n=5) compared to CEA measurements (right).

FIG. 6D is a graph showing improved sensitivity and specificity is reflected in improved AUC values on the receiver operator curve with the macrophage sensor (0.914, 95% CI 0.738-1.00, p=0.019) compared to the endogenous biomarker (0.829, 95% CI 0.590-1, p=0.062). * indicates statistical significance at p<0.05. AUC denotes area under the curve.

FIG. 6E is a hybrid scatter plot/box graph showing plasma concentration of cfDNA was not significantly increased above healthy levels until subcutaneous CT26 tumor volumes reached 1500-2000 mm³.

FIG. 6F illustrates yes/no plots showing neither assayed mutation was detectable by qPCR in mouse plasma cfDNA (n=23, left; n=28, right) until tumors reached volumes of greater than about 1300 mm³. Downward bars indicate a tumor-bearing mouse wherein the mutation was not detected in plasma cfDNA and vertical bars indicate that the mutation was detected.

FIGS. 7A-7C illustrate bone marrow-derived macrophage purity and electroporation efficiency.

FIG. 7A is a FACS plot showing harvested BMDMs exhibited 97.4% purity by F4/80 staining after 5 days of activation with 10 ng/mL murine colony stimulating factor (M-CSF).

FIG. 7B is a FACS plot showing BMDMs were electroporated with the pARG1-Gluc reporter plasmid with an efficiency of approximately 40% as quantified by flow cytometry.

FIG. 7C is a FACS plot showing BMDMs were electroporated with the pARG1-Gluc reporter plasmid with an efficiency of >80% and viability -60% as quantified by flow cytometry.

FIG. 8 illustrates a pARG1-Gluc Reporter Plasmid Map. The pARG1-Gluc construct contains the Gaussia Dura Luciferase immediately downstream of the 3780 base pair ARG1 enhancer/promoter sequence. The construct also contains the gene for enhanced Green Fluorescent protein (eGFP) under the control of the constitutive CMV promoter for cell sorting and determining transfection or electroporation efficiency.

FIG. 9 is a FACS plot showing VivoTrack 680 Labeling of RAW264.7 Macrophages. Uniform labeling of macrophages (blue) was observed with 4-5 orders of magnitude of fluorescence above unstained macrophages (red).

FIGS. 10A and 10B illustrate the detection of metastatic breast cancer using transiently transfected bone marrow-derived macrophages.

FIG. 10A is a scatter plot showing RLU values from plasma of mice bearing metastatic breast cancer (n=5) are significantly elevated (AUC=0.920, 95% CI 0.739-1.00, p=0.028) above healthy control (n=5) upon intravenous injection of BMDM sensor.

FIG. 10B is a series of digital photographs showing BLI of activated BMDM (white circles) and metastatic nodules reveals co-localization in the hind limb. Left and right radiance scales apply to the activated macrophages and tumor signals respectively. RLU, relative luminescence units; AUC, area under the curve.

FIG. 11 illustrates lung microtumors in a model of metastatic breast cancer. One week after intravenous injection of 4T1 cells, disease burden remains localized to the lungs as visualized by BLI (left). Ex vivo examination of the lungs also reveals non-elevated microtumors lining the lung pleura (right). Scale bars measure 1 cm.

FIGS. 12A-12C illustrate macrophage sensor optimization in subcutaneous localized model of colorectal cancer.

FIG. 12A is a graph illustrating tumor volumes measured by digital caliper are well-correlated with tumor volumes estimated by BLI (r²=0.918). Dashed lines show 95% confidence interval of the linear regression.

FIG. 12B is a graph illustrating the engineered macrophage sensor was unable to detect visibly necrotic tumors with volumes greater than 1500 mm³, possibly due to poor infiltration of the sensor into the avascular tumor cores.

FIG. 12C is a scatter plot showing detection of 50-200 mm³ localized subcutaneous tumors, elevated plasma Gluc compared to healthy controls was apparent 24 hours after macrophage sensor injection but signal declined in subsequent days in both healthy and tumor bearing mice.

FIG. 13 shows a pair of box graphs showing lactic acid induces ARG1 expression in macrophages; 100 mM lactic acid induces expression of FIZZ1 and ARG1 mRNA in both bone marrow derived (left) and RAW264.7 (right) macrophages 24 hours after stimulation. * indicates statistical significance at p<0.05, ** indicates statistical significance at p<0.01. Error bars depict standard error of the mean. BMDM, bone marrow-derived macrophage.

FIGS. 14A and 14B show a flow cytometry gating strategy for macrophage sorting.

FIG. 14A illustrates a series of FACS plots showing a flow cytometry gating strategy for adoptively transferred (VT680+) and native (VT680−) macrophages in tumor, spleen, lung, and liver. Populations for CD11b+F4/80+ cells were well-separated in both tissues and adoptively transferred macrophages were gated based on a fluorescence minus one control. FMO, fluorescence minus one.

IG. 14B illustrates a series of pie charts showing average fractional makeup of administered (VT680+) vs. endogenous (VT680−) macrophages present in various tissues five days after adoptive transfer.

FIG. 15 illustrates immunofluorescence of macrophage localization relative to hypoxia in CT26 tumors; immunofluorescence micrographs of CT26 tumors from mice injected with fluorescently labeled bone marrow derived monocytes (bottom) reveals co-localization of macrophages with regions of hypoxia. Immunofluorescence of a CT26 tumor from mice not injected with pimonidazole and injected with non-fluorescently labeled bone marrow derived monocytes (top) does not show any signal in the green or red channels confirming specificity. Images are shown at 10x magnification and scale bars measure 250 μm. CB640, CellBrite 640; BMDM, bone marrow derived monocytes.

FIG. 16 illustrates the effect of intravenously injected macrophage sensor on tumor progression; plots showing Intravenous injection of BMDM sensor in subcutaneous tumor-bearing mice (n=4) leads to an initial regression (Day 4, p=0.058) of tumor volume relative to vehicle injected mice (n=3) followed by resumption of exponential growth. Left plot shows growth of individual tumors and right plot shows average tumor volumes. Mice were sacrificed upon tumors exceeding 15 mm in any dimension and average tumor volumes in right plot are only shown for time points in which all mice in a group were still alive. Error bars depict standard error of the mean. BMDM, bone marrow derived macrophage.

FIG. 17 is a pair of graphs illustrating the deletion mutation limit of detection with locked nucleic acid probes. Real-time qPCR amplification plots of CT26 and wildtype Balb/c genomic DNA show that the chromosome 7 (left) and 19 (right) deletions can be detected at allele frequencies of 0.1% and 1% respectively. Each condition is shown in triplicate. RFU, relative fluorescence units; AF, allele frequency.

FIGS. 18A-18D shows the nucleic acid sequence SEQ ID NO 1.

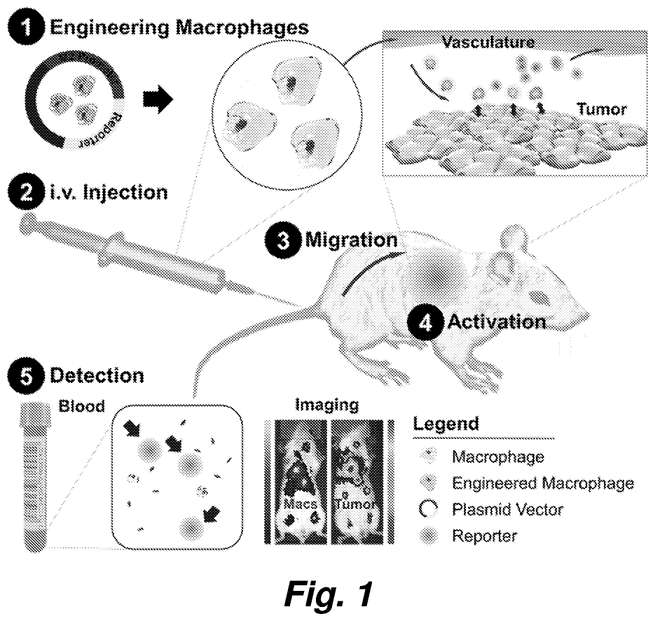

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “ includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Numerical ranges recited herein by endpoints include all numbers and fractions subsumed within that range (e.g. 1 to 5 includes 1, 1, 5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made.

Prior to describing the various embodiments, the following definitions are provided and should be used unless otherwise indicated.

### Abbreviations

SEAP, Secreted Embryonic Alkaline Phosphatase; MRI, magnetic resonance imaging; BLI, bioluminescence imaging; ROI, region of interest; AUC, area under the curve; RG, reporter gene; TS, tumor-specific; Gluc, Gaussia luciferase; CTCs, circulating tumor cells, ctDNA, circulating tumor DNA; HSV1-tk, HSV1-thymidine kinase; hNIS, human sodium iodide symporter; ACT, adoptive cell transfer; RLU, relative luminescence units; BMDM; bone marrow-derived macrophage.

### Definitions

The term “adoptive cell transfer (ACT)” as used herein refers to the transfer of cells into a patient. The cells may have originated from the patient or from another individual. In autologous cancer immunotherapy, T cells are extracted from the patient, genetically modified and cultured in vitro and returned to the same patient.

The term “autologous” as used herein refers to a cell or population of cells isolated or originating from an individual animal or human and then returned to the same individual. The cells may have been genetically modified or cultured before returning to the individual.

The term “biofluid” as used herein refers to a biological fluid sample encompasses a variety of fluid sample types obtained from an individual and can be used in a diagnostic or monitoring assay. The definition encompasses blood total or serum), cerebral spinal fluid (CSF), saliva, tears, sputum, breath, urine and other liquid samples of biological origin. The definition also includes samples that have been manipulated in any way after their procurement, such as by treatment with reagents, solubilization, or enrichment for certain components, such as proteins or polynucleotides.

The term “blood sample” as used herein is a biological sample which is derived from blood, preferably peripheral (or circulating) blood. A blood sample may be, for example, whole blood, plasma, serum, or a solubilized preparation of such fluids wherein the cell components have been lysed to release intracellular contents into a buffer or other liquid medium.

The term “bioluminescence” as used herein refers to a type of chemiluminescent, emission of light by biological molecules, particularly proteins. The essential condition for bioluminescence is molecular oxygen, either bound or free in the presence of an oxygenase, a luciferase, which acts on a substrate, a luciferin in the presence of molecular oxygen and transforms the substrate to an excited state, which upon return to a lower energy level releases the energy in the form of light.

The term “biomarker” as used herein refers to an antigen such as, but not limited to, a peptide, polypeptide, protein (monomeric or multimeric) that may be found on the surface of a cell, an intracellular component of a cell, or a component or constituent of a biofluid such as a soluble protein in a serum sample and which is a characteristic that is objectively measured and evaluated as an indicator of a tumor or tumor cell. The presence of such a biomarker in a biofluid or a biosample isolated from a subject human or animal can indicate that the subject is a bearer of a pathology (e.g. cancer). A change in the expression of such a biomarker may correlate with an increased risk of disease or progression, or predictive of a response of a disease to a given treatment.

The term “cancer”, as used herein, shall be given its ordinary meaning, as a general term for diseases in which abnormal cells divide without control. In particular, cancer refers to angiogenesis related cancer. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different types of cells within it, with differing processes that have gone awry. Solid tumors may be benign (e.g. non-cancerous), or malignant (e.g. cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties such as increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

Benign tumors have less of a tendency to invade and are less likely to metastasize. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

The term “cell or population of cells” as used herein refers to an isolated cell or plurality of cells excised from a tissue or grown in vitro by tissue culture techniques. Most particularly, a population of cells refers to cells in vivo in a tissue of an animal or human.

The terms “coding sequence” and “encodes a selected polypeptide” as used herein refer to a nucleic acid molecule that is transcribed (in the case of DNA) and translated (in the case of mRNA) into a polypeptide, for example, when the nucleic acid is present in a living cell (in vivo) and placed under the control of appropriate regulatory sequences (or “control elements”).

The term “control element” as used herein refers to, but is not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

The term “cytokine” is a generic term for proteins released by one cell population, which act on another cell population as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; placental growth factor, transforming growth factors (TGFs) such as TGF-alpha and TGF-beta; insulin-like growth factor-1 and -11; erythropoietin (EPO); osteoinductive factors; interferons such as interferon-alpha, -beta and -gamma colony stimulating factors (CSFs) such as macrophage-CSF (M-CSF); granulocyte macrophage-CSF (GM-CSF); and granulocyte-CSF (G-CSF); interleukins (ILs) such as IL-1, IL-2, IL-3, IL-4, IL-5, IL-6, IL-7, IL-8, IL-9, IL-10, IL-11, IL-12, IL-13, IL-15, IL-18, IL-21, IL-22, IL-23, and IL-33; a tumor necrosis factor such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

The term “delivering to a cell” as used herein refers to the direct targeting of a cell with a small molecule compound, a nucleic acid, a peptide or polypeptide, or a nucleic acid capable of expressing an inhibitory nucleic acid or polypeptide by systemic targeted delivery for in vivo administration, or by incubation of the cell or cells with the effector ex vivo or in vitro.

The term “detectable agent” refers to refers to a molecule (e.g. small molecule, peptide, protein RNA, DNA) that is detectable by any assay designed to specifically detect relative or absolute concentration of such agent. In some embodiments, the detectable agent is a polypeptide exogenous to the immune cell to which it is introduced. The detectable agent may be detectable by non-invasive imaging methods such as MRI imaging, PET imaging, SPECT imaging, and luminescence imaging including, but not intended to be limiting, a photoacoustic reporter, a bioluminescent reporter, an autofluorescent reporter, a chemiluminescent reporter, a luminescent reporter, or a colorimetric reporter. Suitable MRI reporter genes include e.g. those encoding creatine kinase; tyrosinase; transferrin receptor; ferritin; Mag A. PET imaging reporter genes include, but are not limited to such as Herpes simplex virus 1 thymidine kinase (HSV1-tk); hypoxanthine phosphoribosyl transferase; L-amino acid decarboxylase; dopamine 2 receptor (D2R, including the mutant D2RA80); somatostatin receptor; estrogen receptor (hERL); dopamine transporter; sodium iodide symporter; catecholamine transporter; β-galactosidase. PET/SPECT imaging reporter genes include, but are not limited to, Herpes simplex virus Type 1 thymidine kinase and multiple optimized mutants, such as HSV1-sr39tk; dopamine type 2 receptor; sodium iodide symporter; somatostatin type 2 receptor; human norepinephrine transporter; human estrogen receptor a; mutants of human deoxycytidine kinase; and recombinant carcinoembryonic antigen. Bioluminescence reporter genes include, but are not limited to, firefly luciferase (fl); Gaussia luciferase (Gluc); synthetic Renifia luciferase (hrl); Enhanced Green Fluorescence protein (egfp); Red Fluorescence Protein (rfp); monomeric Red Fluorescence Protein (mrfp1), and the like. It is further possible for the reporter genes suitable for incorporation into the genetic constructs of the disclosure to provide multi-modality methods of imaging.

The term “expression cassette” as used herein refers to any nucleic acid construct capable of directing the expression of any RNA transcript including gene/coding sequence of interest as well as non-translated RNAs, such as shRNAs, microRNAs, siRNAs, anti-sense RNAs, and the like. Such cassettes can be constructed into a “vector,” “vector construct,” “expression vector,” or “gene transfer vector,” in order to transfer the expression cassette into target cells. Thus, the term includes cloning and expression vehicles, as well as plasmids and viral vectors.

The term “expression vector” as used herein refers to a nucleic acid useful for expressing the DNA encoding the protein used herein and for producing the protein. The expression vector is not limited as long as it expresses the gene encoding the protein in various prokaryotic and/or eukaryotic host cells and produces this protein. When yeast, animal cells, or insect cells are used as hosts, an expression vector preferably comprises, at least, a promoter, an initiation codon, the DNA encoding the protein and a termination codon. It may also comprise the DNA encoding a signal peptide, enhancer sequence, 5′-and 3′-untranslated region of the gene encoding the protein, splicing junctions, polyadenylation site, selectable marker region, and replicon. The expression vector may also contain, if required, a gene for gene amplification (marker) that is usually used.

A promoter/operator region to express the protein in bacteria comprises a promoter, an operator, and a Shine-Dalgarno (SD) sequence (for example, AAGG). For example, when the host is Escherichia, it preferably comprises Trp promoter, lac promoter, recA promoter, lambda.PL promoter, b 1 pp promoter, tac promoter, or the like. When the host is a eukaryotic cell such as a mammalian cell, examples thereof are SV40-derived promoter, retrovirus promoter, heat shock promoter, and so on. As a matter of course, the promoter is not limited to the above examples. In addition, using an enhancer is effective for expression. A preferable initiation codon is, for example, a methionine codon (ATG). A commonly used termination codon (for example, TAG, TAA, and TGA) is exemplified as a termination codon. Usually, used natural or synthetic terminators are used as a terminator region. An enhancer sequence, polyadenylation site, and splicing junction that are usually used in the art, such as those derived from SV40, can also be used. A selectable marker usually employed can be used according to the usual method. Examples thereof are resistance genes for antibiotics, such as tetracycline, ampicillin, or kanamycin.

The expression vector used herein can be prepared by continuously and circularly linking at least the above-mentioned promoter, initiation codon, DNA encoding the protein, termination codon, and terminator region to an appropriate replicon. If desired, appropriate DNA fragments (for example, linkers, restriction sites, and so on) can be used by a method such as digestion with a restriction enzyme or ligation with T4 DNA ligase. Transformants can be prepared by introducing the expression vector mentioned above into host cells.

The terms “heterologous sequence” or a “heterologous nucleic acid”, as used herein refer to a nucleic acid that originates from a source foreign to the particular host cell, or, if from the same source, is modified from its original form. Thus, a heterologous expression cassette in a cell is an expression cassette that is not endogenous to the particular host cell, for example by being linked to nucleotide sequences from an expression vector rather than chromosomal DNA, being linked to a heterologous promoter, being linked to a reporter gene, etc.

The term “inflammation” as used herein refers to acute and chronic disorders where homeostasis is disrupted by an abnormal or dysregulated inflammatory response. These conditions are initiated and mediated by a number of inflammatory factors, including oxidative stress, chemokines, cytokines, breakage of blood/tissue barriers, autoimmune diseases or other conditions that engage leukocytes, monocytes/macrophages or parenchymal cells that induce excessive amounts of pro-cell injury, pro-inflammatory/disruptors of homeostasis mediators. These diseases occur in a wide range of tissues and organs and are currently treated, by anti-inflammatory agents such as corticosteroids, non-steroidal anti-inflammatory drugs, TNF modulators, COX-2 inhibitors, etc.

The term “in vivo imaging” as used herein refers to methods or processes in which the structural, functional, or physiological state of a living being is examinable without the need for a life-ending sacrifice.

The term “luciferase” as used herein refers to oxygenases that catalyze a light emitting reaction. For instance, bacterial luciferases catalyze the oxidation of flavin mononucleotide and aliphatic aldehydes, whereupon the reaction produces light. Another class of luciferases, found among marine arthropods, catalyzes the oxidation of cypridina luciferin, and another class of luciferases catalyzes the oxidation of coleoptera luciferin. Thus, “luciferase” refers to an enzyme or photoprotein that catalyzes a bioluminescent reaction. The luciferases such as firefly and Renilla luciferases are enzymes that act catalytically and are unchanged during the bioluminescence generating reaction. The luciferase photoproteins, such as the aequorin and obelin photoproteins to which luciferin is non-covalently bound, are changed by release of the luciferin, during bioluminescence generating reaction. The luciferase is a protein that occurs naturally in an organism or a variant or mutant thereof, such as a variant produced by mutagenesis that has one or more properties, such as thermal or pH stability, that differ from the naturally-occurring protein. Luciferases and modified mutant or variant forms thereof are well known. Reference, for example, to “Renilla luciferase” means an enzyme isolated from member of the genus Renilla or an equivalent molecule obtained from any other source, such as from another Anthozoa, or that has been prepared synthetically. Reference to “Gaussia luciferase” means an enzyme isolated from member of the genus Gaussia.

“Bioluminescent protein” refers to a protein capable of acting on a bioluminescent initiator molecule substrate to generate or emit bioluminescence.

“Bioluminescent initiator molecule” is a molecule that can react with a bioluminescent donor protein to generate bioluminescence. The bioluminescence initiator molecule includes, but is not limited to, coelenterazine, analogs thereof, and functional derivatives thereof. Derivatives of coelenterazine include, but are not limited to, coelenterazine 400a, coelenterazine cp, coelenterazine f, coelenterazine fcp, coelenterazine h, coelenterazine hcp; coelenterazine ip, coelenterazine n, coelenterazine O, coelenterazine c, coelenterazine c, coelenterazine i, coelenterazine icp, coelenterazine 2-methyl, benzyl-coelenterazine bisdeoxycoelenterazine, and deep blue coelenterazine (DBC) (described in more detail in U.S. Pat. Nos. 6,020,192; 5,968,750 and 5,874,304).

The term “macrophage” as use herein refers to classically-activated macrophages (M1 macrophages) and alternatively-activated macrophages (M2 macrophages). Martinez et al., Annu. Rev. Immunol. 27: 451-483 (2009). Generally, M1 macrophages exhibit potent anti-microbial properties, reminiscent of type 1 T-helper lymphocyte (Th1) responses. In contrast, M2 macrophages promote type 2 T-helper lymphocyte (Th2)-like responses, secrete less pro-inflammatory cytokines, and assist resolution of inflammation by trophic factor synthesis and phagocytosis. Mosser et al., Nature Rev. 8:958-969 (2008). M2 macrophages can be further divided into three distinct subclasses, i.e., M2a, M2b, and M2c, defined by specific cytokine profiles. Mantovani et al., Trends Immunol. 25:677-686 (2004). While M2 macrophages are generally characterized by low production of pro-inflammatory cytokines, such as IL-12, and high production of anti-inflammatory cytokines such as IL-10, M2b macrophages retain high levels of inflammatory cytokine production, such as TNF-α and IL-6 (Mosser, J. Leukocyte Biol. 73:209-212 (2003)).

Macrophages can be polarized by their microenvironment to assume different phenotypes associated with different stages of inflammation and healing. Stout et al., J. Immunol. 175:342-349 (2005). Certain macrophages are indispensible for wound healing. They participate in the early stages of cell recruitment and of tissue defense, as well as the later stages of tissue homeostasis and repair. (Pollard, Nature Rev. 9:259-270 (2009)). Macrophages derived from peripheral blood monocytes have been used to treat refractory ulcers. Danon et al., Exp. Gerontol. 32:633-641 (1997); Zuloff-Shani et al., Transfus. Apher. Sci. 0:163-167 (2004), each of which is incorporated herein by reference as if set forth in its entirety.

The term “modify the level of gene expression” as used herein refers to generating a change, either a decrease or an increase in the amount of a transcriptional or translational product of a gene. The transcriptional product of a gene is herein intended to refer to a messenger RNA (mRNA) transcribed product of a gene and may be either a pre- or post-spliced mRNA. Alternatively, the term “modify the level of gene expression” may refer to a change in the amount of a protein, polypeptide or peptide generated by a cell as a consequence of interaction of an siRNA with the contents of a cell. For example, but not limiting, the amount of a polypeptide derived from a gene may be reduced if the corresponding mRNA species is subject to degradation as a result of association with an siRNA introduced into the cell.

The term “modulate” refers to the activity of a composition to affect (e.g., to promote or retard) an aspect of cellular function, including, but not limited to, cell growth, proliferation, apoptosis, and the like.

The terms “nucleic acid,” “nucleic acid sequence,” or “oligonucleotide” also encompass a polynucleotide. A “polynucleotide” refers to a linear chain of nucleotides connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides linked by a phosphodiester backbone. A “modified polynucleotide” refers to a polynucleotide in which natural nucleotides have been partially replaced with modified nucleotides.

The term “operably linked” as used herein refers to an arrangement of elements wherein the components so described are configured so as to perform their usual function. Thus, a given promoter that is operably linked to a coding sequence (e.g., a reporter expression cassette)is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The term “primer” as used herein refers to an oligonucleotide complementary to a DNA segment to be amplified or replicated. Typically primers are used in PCR. A primer hybridizes with (or “anneals” to) the template DNA and is used by the polymerase enzyme as the starting point for the replication/amplification process. By “complementary” it is meant that the primer sequence can form a stable hydrogen bond complex with the template.

The term “promoter” is a DNA sequence that directs the transcription of a polynucleotide. Typically a promoter can be located in the 5′ region of a polynucleotide to be transcribed, proximal to the transcriptional start site of such polynucleotide. More typically, promoters are defined as the region upstream of the first exon; more typically, as a region upstream of the first of multiple transcription start sites. Frequently promoters are capable of directing transcription of genes located on each of the complementary DNA strands that are 3′ to the promoter. Stated differently, many promoters exhibit bidirectionality and can direct transcription of a downstream gene when present in either orientation (i.e. 5′ to 3′ or 3′ to 5′ relative to the coding region of the gene). Additionally, the promoter may also include at least one control element such as an upstream element. Such elements include upstream activator regions (UARs) and optionally, other DNA sequences that affect transcription of a polynucleotide such as a synthetic upstream element. Promoters advantageous for use in the embodiments of the disclosure include, but are not limited to an AKT1 promoter, a versican promoter, a MIF promoter, a Ym1 promoter, a CD206 promoter, a FIZZ1 promoter, a DC-SIGN promoter, a CD209 promoter, a MGL-1 promoter, a Dectin -1 promoter, a CD23 promoter, a galectin-3 promoter, a Mer tyrosine kinase promoter, an AXL receptor protein promoter, a GAS-6 promoter, a NOS-2 promoter, a CD68 promoter, a CD86 promoter, a CCL18 promoter, a CD163 promoter, a MMR/CD206 promoter, a CD200R promoter, a TGM2 promoter, a DecoyR promoter, an IL-1 R II promoter, an IL-10 promoter, a TGF-beta promoter, an IL-1ra promoter, a CCL17 promoter, a CCL2 promoter, or a CCL24 promoter.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “qPCR” refers to a real-time polymerase chain reaction, also called quantitative real time polymerase chain reaction (Q-PCR/qPCR/qrt-PCR) which is used to amplify and simultaneously detect the quantity of a targeted DNA molecule. The quantity can be expressed as either a number of copies or a relative amount normalized to the input DNA. Detection proceeds as the reaction progresses in real time unlike standard PCR, where the product of the reaction is detected at its end point. Two common methods for detection of products in real-time PCR are: (1) non-specific fluorescent dyes that intercalate with any double-stranded DNA, and (2) sequence-specific oligonucleotides that are labeled with a fluorescent reporter and permit detection after hybridization to their complementary DNA target.

The term “transformation,” refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods described in various general and more specific references that are cited and discussed throughout the present specification. See e.g., Sambrook et al. (1989) Molecular Cloning: A Laboratory Manual (2d ed., Cold Spring Harbor Laboratory Press, Cold Spring Harbor, N.Y.).

The term “vector” is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, additional embodiments include such other forms of expression vectors, such as viral vectors (e.g., replication defective retroviruses, adenoviruses and adeno-associated viruses), which serve equivalent functions.

### Discussion

Current endogenous biomarker-based cancer tests lack the sensitivities and specificities required to reliably detect disease in its earliest malignant or pre-malignant stage, at least in part due to rapid biomarker clearance from the blood, high background signal from healthy tissue or confounding disease states, and relatively low concentrations in peripheral circulation.

An alternative diagnostic strategy to the traditional reliance on detection and quantification of endogenous biomarkers for disease detection is the systemic delivery of exogenous probes that can be selectively activated in the presence of disease and subsequently generate a signal that is detectable by either imaging or sampling of body fluids. As they are not limited by, for example, the immunological detectability of an endogenous biomarker that happens to be associated with a disease, approaches using these activatable exogenous probes to detect disease likely have higher sensitivities and signal-to-noise ratios than approaches using endogenous biomarkers. To date, synthetic biomarker strategies have been limited by either (i) biocompatibility of the probe, (ii) efficient delivery of the probe to diverse sites of disease, (iii) the ability of the probe to assay multiple features of a biochemically complex disease environment, or (iv) the ability to provide both spatial location information as well as yes/no answers from patient samples.

The present disclosure encompasses embodiments of genetically engineered immune cells that provide a new cell-based in vivo sensor platform for the ultrasensitive detection of disease. The adoptive transfer of syngeneic macrophages engineered to produce a synthetic biomarker upon adopting a ‘tumor-associated’ metabolic profile. In some cases, this platform allows detection of tumors as small as 25-50 mm³, effectively tracks the immunological response in a model of inflammation, and is more sensitive than a clinically used biomarker of tumor recurrence. This technology establishes a highly biocompatible and clinically translatable approach to early cancer detection and provides a conceptual framework for the use of engineered immune cells for the monitoring of many disease states including, but not limited to, cancer.

With endogenous biomarkers still unable to detect disease in its earliest stages or reliably localize site of origin, disease-activatable probes continue to be a promising approach to early disease detection, localization, and monitoring. The present disclosure provides a novel platform technology for early disease detection using engineered immune cells as the diagnostic sensors. By leveraging disease-specific metabolic alteration in macrophages, it has now been show that an immune cell sensor can detect tumors at least as small as 4 mm in diameter and exhibit greater sensitivity than a clinically used biomarker of tumor recurrence/occurrence. Tumor volumes of 25-50 mm³ and diameters detectable by the engineered macrophages of the disclosure are below the limits of detection of current clinical PET molecular imaging (approximately 200 mm³ and 7 mm respectively), indicating that macrophages can migrate to and alter their metabolic state in early tumorigenesis and are advantageous as diagnostic sensors for small tumors. It has further been shown through models of inflammation that such a sensor can both potentially achieve high specificity and be usefully applied to monitoring of disease states other than cancer.

Accordingly, the present disclosure encompasses embodiments of engineered (e.g. genetically modified), macrophage cells which may or may not have been originally isolated from a patient. In some cases, the genetic modification is achieved by introducing into the cells a gene expression vector encoding a detectable agent (e.g. detectable polypeptide or synthetic RNA) under the expression control of a gene promoter/enhancer inducible by a tumor-specific metabolic factor. The gene promoter/enhancer inducible by a tumor-specific metabolic factor may comprise regulatory sequences of ARG1, which is induced in M2 macrophages when the macrophages are in the presence of a tumor. Cytokines and other tumor-mediated factors such as an increase in acidity may induce the promoter activity of ARG1.

Thus, it has been found, for example, that placing a nucleic acid encoding a detectable agent under the expression control of ARG1 results in the expression of the detectable agent when the engineered macrophages are in contact with, or in close proximity to, a tumor. It has further been found that selection of a detectable agent that can be secreted from the macrophages into the blood stream allows for detection of the agent in a blood sample, thereby indicating the likely presence of a tumor in the patient. While detection of the agent in a blood sample advantageously allows for a rapid and economical approach for determining the presence of a tumor in a patient, a percentage of the expressed product is also retained within the macrophages in contact with the tumor. This can further allow for the spatial detection of the tumor within the patient's body.

In one embodiment of the disclosure, the detectable agent can be Gaussia luciferase (Gluc), and it has been found possible to significantly increase the sensitivity of the detection of tumors compared to other detection methods. However, alternative detectable agents expressible by constructs of the disclosure are also possible. Thus, for example, in some embodiments of the disclosure, the detectable agent can be human chorionic gonadotropin (HCG) or a derivative thereof, secretable alkaline phosphatase (SEAP), or even non-protein biomarkers such as artificial secreted microRNAs can be used. SEAP has many ideal characteristics as a detectable agent. It is an artificial, C-terminal truncated, secretable form of human placental alkaline phosphatase (PLAP) that is only expressed during embryogenesis; thus it is a unique reporter not normally found in the blood and should have near-zero background (Berger et al., (1988) Gene 66: 1-10). Compared to PLAP, SEAP is unusually heat-stable; thus heating samples to 65° C. allows SEAP to be specifically assayed (Bronstein et al., (1994) BioTechniques 17: 172-174, 76-177). Commercial SEAP detection assays are extremely sensitive over at least a 4-log order concentration range, with detection limits in the picogram/ml range. SEAP is also an advantageous protein-based reporter for translation into the clinic since: 1) it has shown effective longitudinal monitoring of non-viral gene transfer in mice and large animals (Brown et al., (2008) Methods Mol. Biol. 423: 215-224); 2) its human origin implies it can have reduced or zero immunogenic potential in patients similar to what has been shown with murine SEAP (muSEAP) in immunocompetent mice (Wang et al., (2001) Gene 279: 99-108); and 3) SEAP has been used in the clinic to monitor antibody levels following administration of an HPV16/18 AS04-adjuvanted vaccine (Kemp et al., (2008) Vaccine 26: 3608-3616).

The engineered immune cell platform of the disclosure described herein overcome many hurdles to clinical translation faced by competing approaches. For example, nanoparticle-based sensors and engineered bacterial sensors have been used to monitor disease processes including cancer, fibrosis, thrombosis, and inflammation in vivo with considerable success. Despite this, their clinical translation has been hindered by unfavorable pharmacokinetics (PK), unreliable delivery to sites of disease, and inherent immunogenicity of the sensors.

In contrast, the presently disclosed engineered macrophage platforms exhibit natural biochemical-mediated responses to sites of pathology and can use a subject's own (e.g. autologous) cells to overcome issues of biocompatibility. Moreover, the ability to generate secreted tumor- or disease-induced biomarkers and use an associated blood test prior to clinical imaging is more economical and can be administered more efficiently than screening tests that require costly and time-consuming clinical imaging for every subject. In addition, most currently described or clinically used artificial sensors are limited to monitoring a single feature of pathology (e.g. over-expression of a protease, or the Warburg effect in the case of ¹⁸FDG PET). In contrast, the cells of the engineered immune cell platform described herein are can be capable of integrating multiple of the complex physical and biochemical cues that make up a disease environment (such as a TME) to alter the expression of metabolic genes. In this way, cell-based sensors can leverage nature's complex genetic and/or biochemical pathways to provide circuitries for improved detection sensitivity and specificity without extensive engineering.

The compositions and methods of the present disclosure have the additional advantage of accommodating a choice of disease state, immune cell subtype, and reporter construct. While the proof-of-concept uses the canonical example of macrophages and the associated M2 phenotype can act as a diagnostic surrogate for cancer, other immune subtypes including T-cells, B-cells, natural killer cells, and dendritic cells have all shown to modulate key metabolic genes in the presence of a tumor microenvironment (TME) and other disease states. Accordingly, such immune cells, which may migrate to a pathology site in a patient, may also be engineered according to the methods of the disclosure to act as tumor or disease-specific cell sensors. T-cells in particular are attractive from a translational standpoint as the diagnostic constructs can be introduced along with a chimeric antigen receptor (CAR) for patients already undergoing CAR T-cell therapy. This would allow dynamic assessment of T-cell metabolism during therapy for detecting residual disease burden or even monitoring clinically actionable phenotypes such as T-cell exhaustion. Further, while Gluc is initially used as a synthetic biomarker because simple and senstive detection methods are available for it (e.g. luciferase assays), other less immunogenic synthetic biomarkers (e.g. secreted placental alkaline phosphatase (SEAP) or secreted synthetic RNAs/microRNAs) can be used.

As demonstrated by the co-localization of a BLI signal from activated macrophages with sites of pathology in our study, spatial information provided by the engineered macrophage platforms described herein can be useful in clinical decision-making. The tracking of adoptively transferred CAR T-cells containing the HSV1-tk reporter gene using PET has been reported. Accordingly, embodiments of the engineered immune cells platform described herein can be adapted by replacing Gluc with HSV1-tk, performing PET to visualize regions of M2 polarization, and observing spatial distribution of the BLI signal to assess the patient's disease status. Clinical precedent for such imaging procedures already exist. For one, indium-111 white blood cell scans, which involve radiolabeling a patient's neutrophils and tracking them in vivo with SPECT, are used to localize potential areas of infection. Alternatively, iron oxide nanoparticles have been used to image tumor-associated macrophages in solid tumors with magnetic resonance imaging (MRI).

While the system described herein may involve personalized development of immune cell probes, there are several options for improving the general applicability of this system. Techniques such as in vivo gene delivery to an immune subset of interest could enable generating diagnostic sensors in situ without the cell isolation and engineering.

### Engineered Cells

Multiple techniques have been established for both transient and stable transfection and of primary somatic cells such as those utilized herein. Transient techniques include delivery of nucleic acids (e.g. DNA plasmids or DNA minicircles bearing synthetic gene elements like the synthetic biomarker constructs described herein) via e.g. lipofection, polyethylenimine(PEI)-mediated transfection, and calcium-phosphate mediated transfection, electroporation, or nucleofection. Stable transfection includes both viral and non-viral techniques. Viral techniques include lentivirus-based, adenovirus-based, or adeno-associated-virus-based transduction with replication-deficient virions engineered to include synthetic gene elements (e.g. the synthetic biomarker constructs described herein). Non-viral techniques include transfection with DNA vectors bearing episomal maintenance elements (e.g. S/MAR containing plasmids or CELiD vectors), transfection and random integration of circular or linear synthetic DNA molecules, and CRISPR-directed cleavage and homology-directed repair using circular or linear synthetic DNA molecules.

Arginase-1 Expression Identifies Tumor-Associated Macrophages For a broadly applicable diagnostic sensor, an immune cell subset can be selected

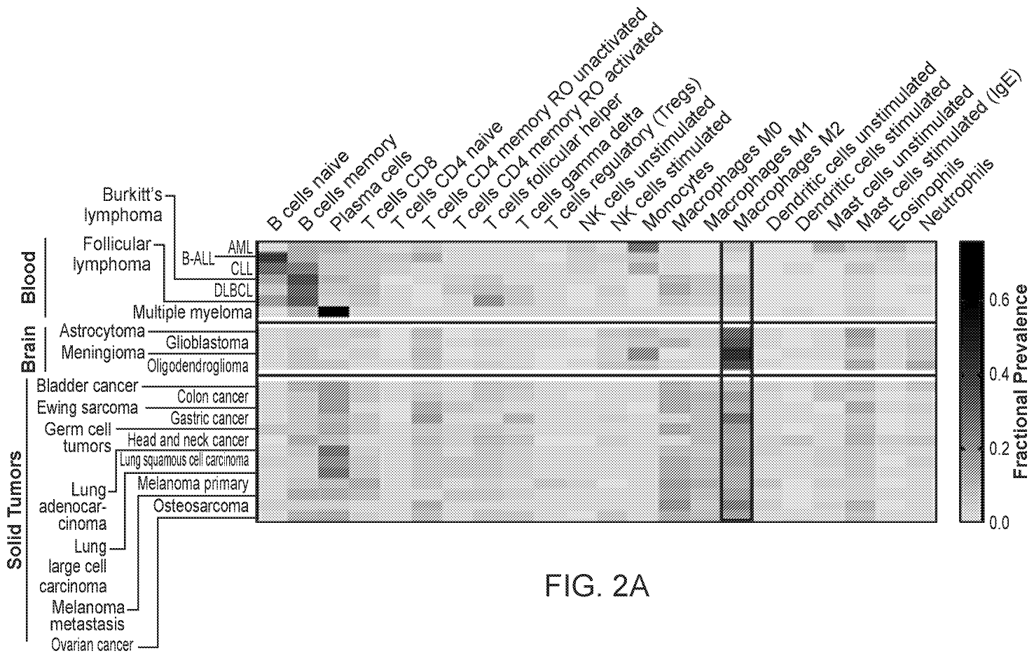

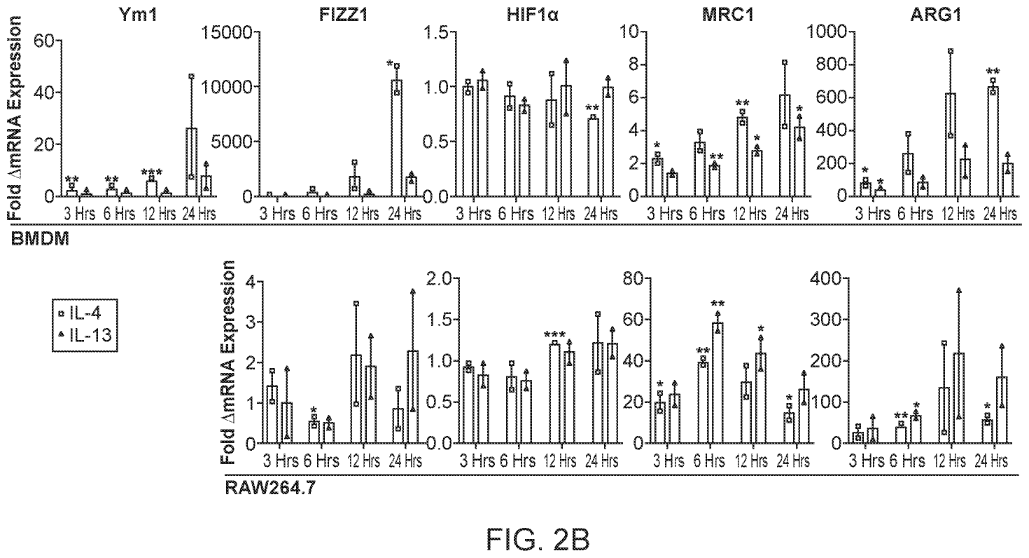

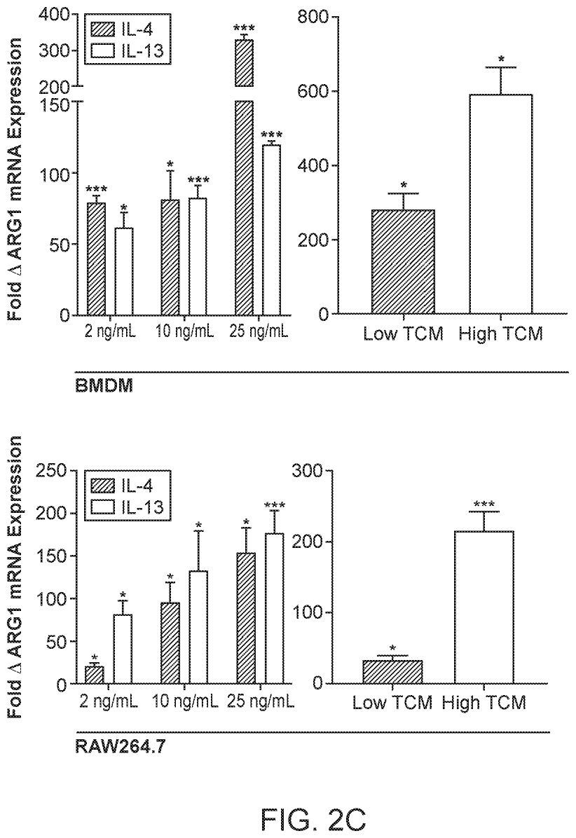

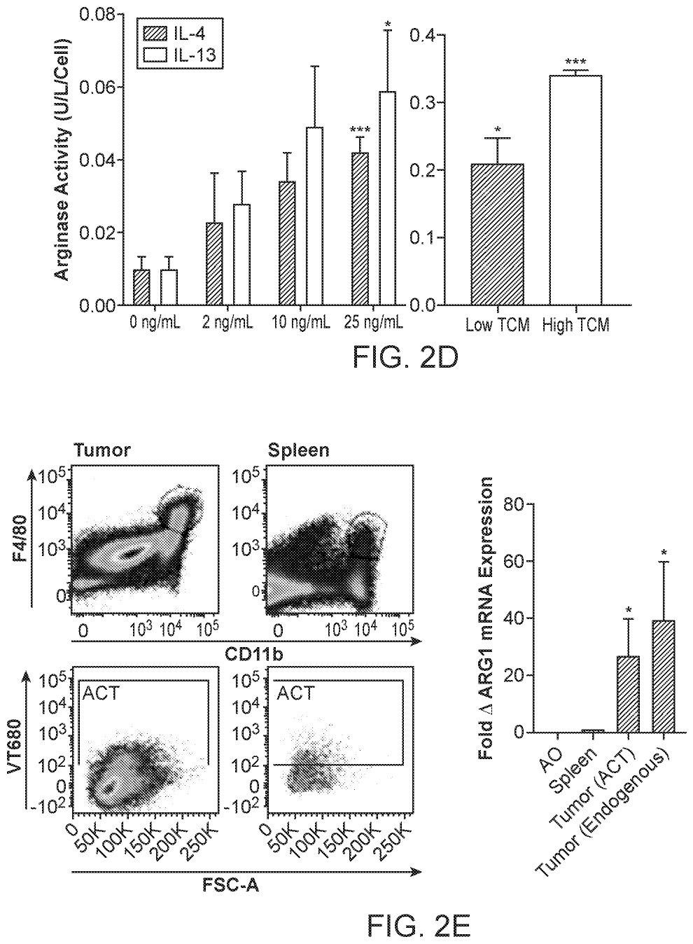

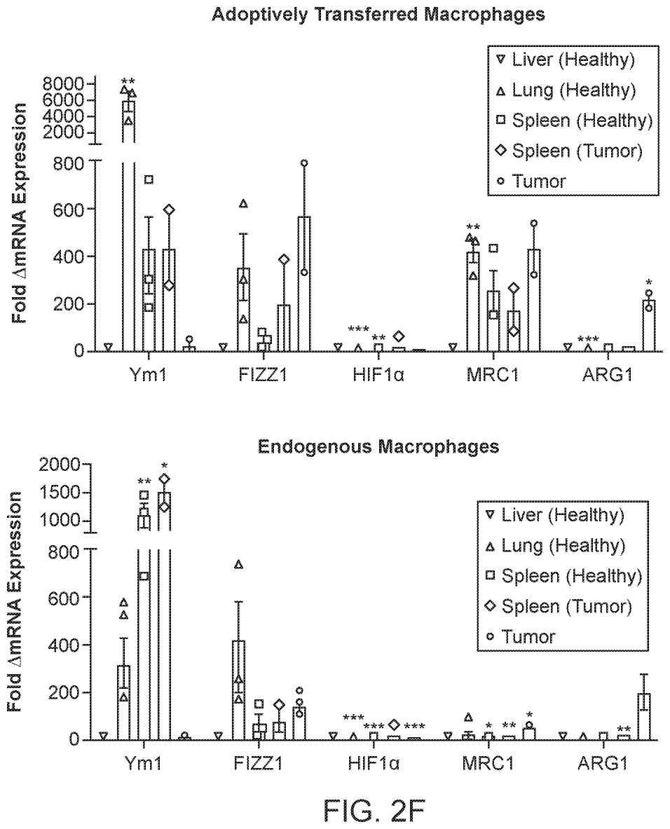

that is widely present across a range of human cancers. Analysis of the fractional prevalence of tumor infiltrating leukocytes across 5,782 tumor specimens from the immune prediction of clinical outcomes from genomic profiles (iPRECOG) dataset showed M2 macrophages are the predominant immune cell population present in the majority of solid tumors with a fractional abundance of up to 0.43 (meningioma) (FIG. 2A), indicating its usefulness as a pan-cancer diagnostic sensor in the platforms described herein.

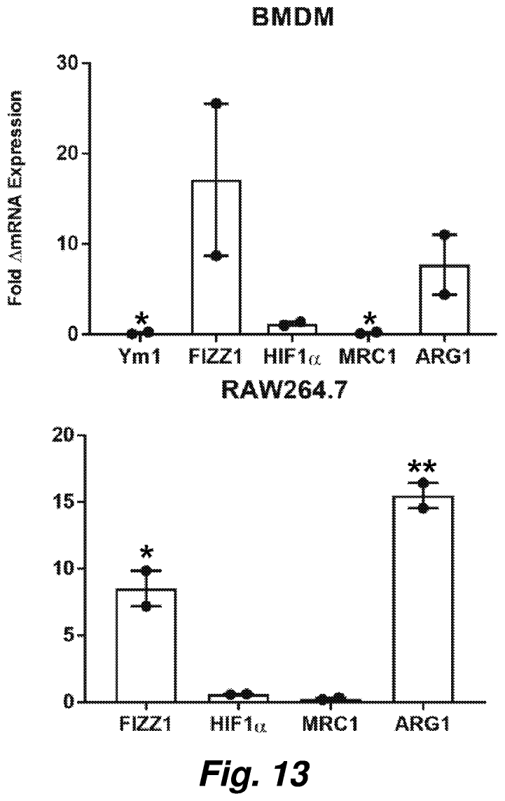

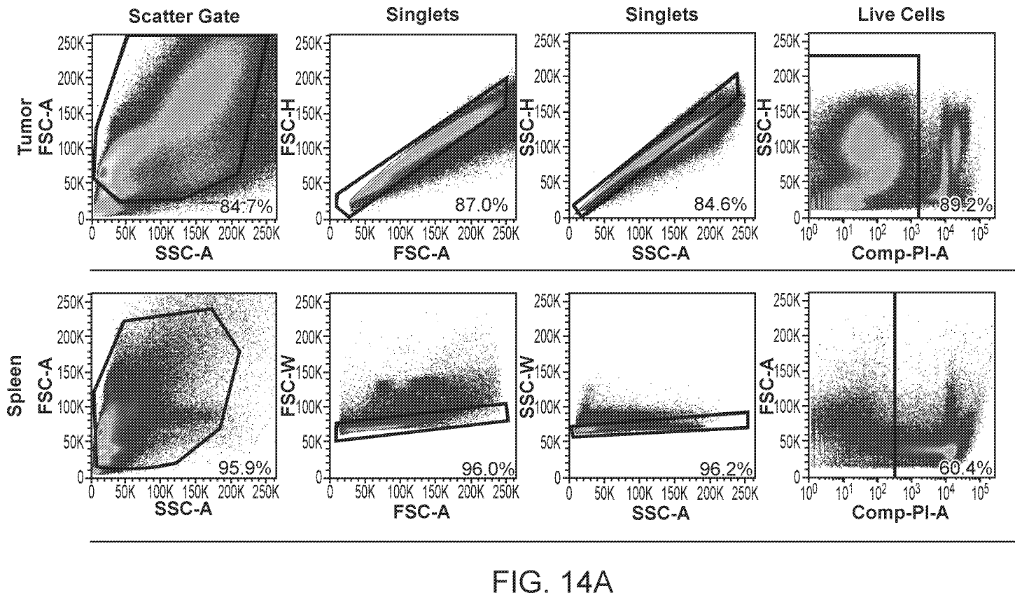

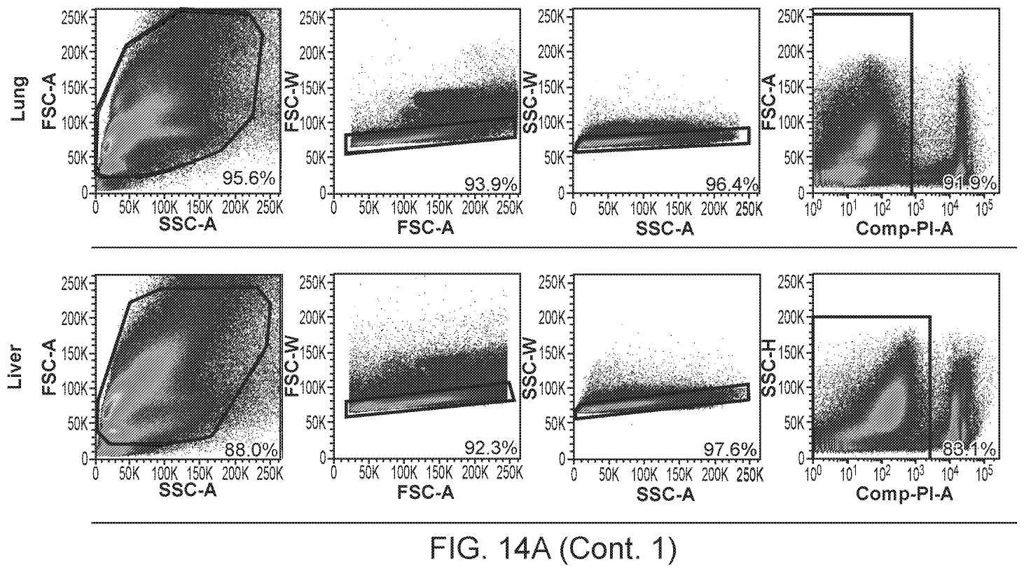

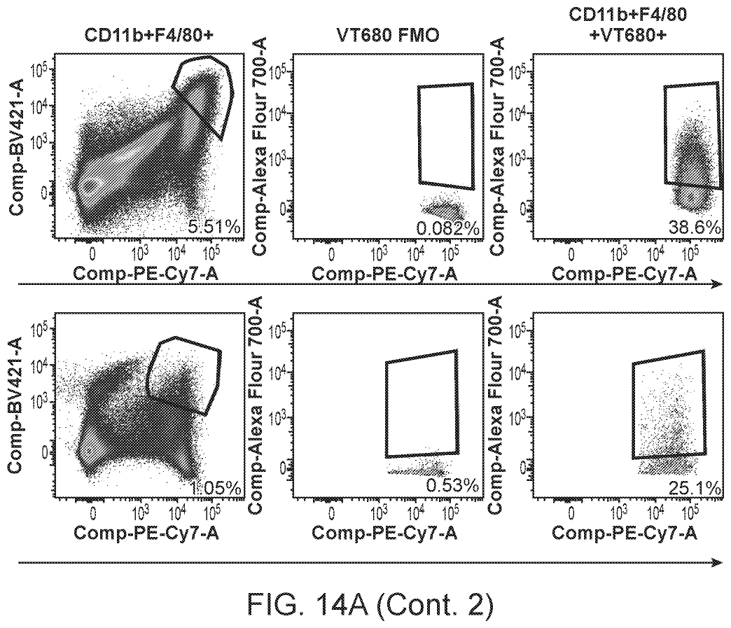

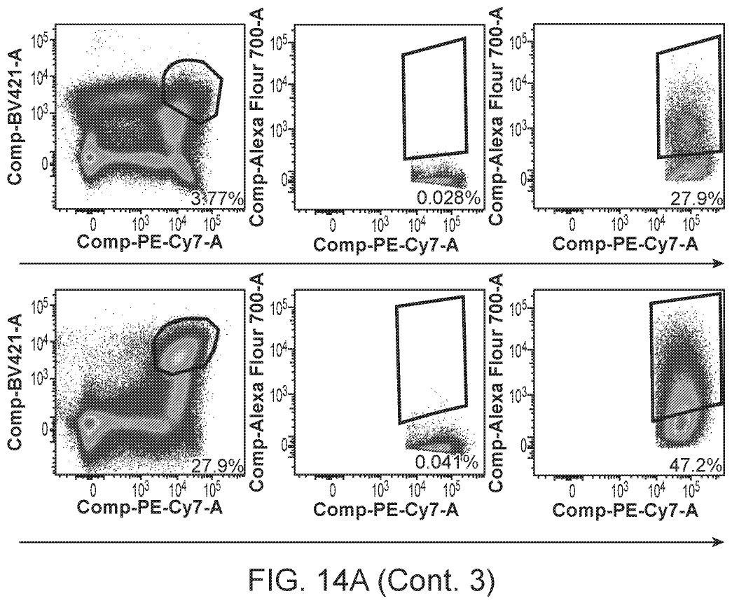

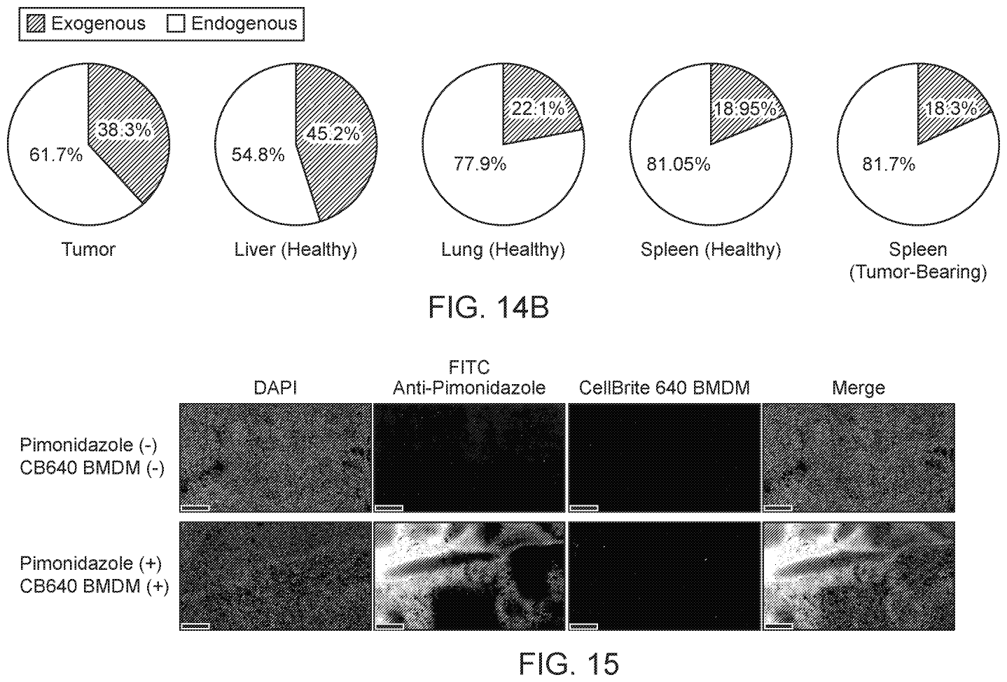

In mice, tumor-associated M2-polarization is characterized by upregulation of gene products (e.g. ARG1) involved in fostering an immunosuppressive microenvironment. It was, therefore, investigated whether ARG1 expression could be a used as a diagnostic surrogate for macrophage encounters with a tumor microenvironment (TME). The TME is a complex niche characterized by acidosis, hypoxia, elevated concentrations of T-helper-2 (Th2) cytokines including IL-4 and IL-13, and tumor-derived cytokines and metabolites. Both BMDMs and the RAW264.7 macrophage cell line exhibited similar concentration-dependent increases in ARG1-specific mRNA expression and activity upon stimulation with IL-4 and IL-13 as measured with quantitative PCR (qPCR) (FIGS. 2B and 2C) and arginase activity assays (FIG. 2D) respectively. This dose-dependent effect was also replicated upon exposure to tumor-conditioned media (TCM) from a CT26 murine colon carcinoma cell line with BMDMs exhibiting upwards of 600±65-fold increase in ARG1 expression (FIGS. 2B and 2C and 2D). This effect is mediated by both tumor-derived cytokines as well as the acidity of TCM. The effect appears to be mediated by tumor derived cytokines and metabolic intermediates including lactic acid (FIG. 13).

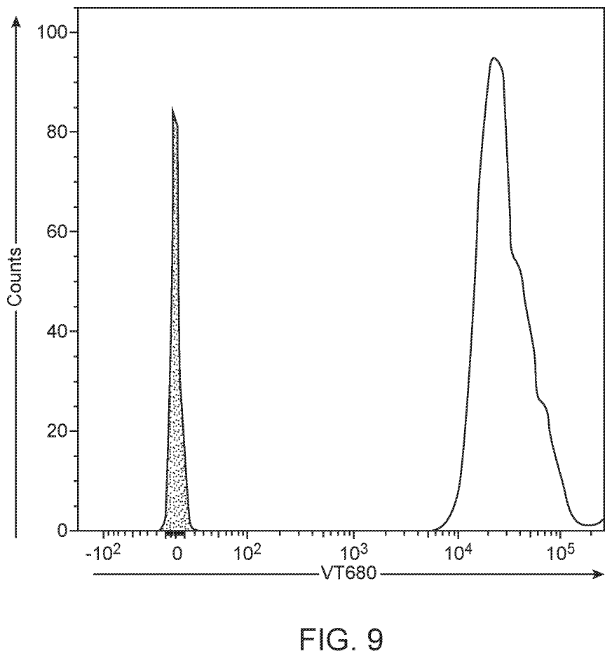

To assess whether adoptively transferred macrophages can take on a tumor-associated phenotype in tumor-bearing mice, RAW264.7 cells were labeled with VivoTrack 680 (VT680) membrane dye prior to intravenous injection into BALB/c mice harboring syngeneic 25-50 mm³ subcutaneous colorectal tumors. Uniform labeling of macrophages was confirmed by flow cytometry (FIG. 9). Five days after injection of the labeled macrophages, the tumors and spleens were harvested, and endogenous (CD11b+, F4/80+, VT680−) and adoptively transferred (CD11b+, F4/80+, VT680+) macrophages were isolated by flow cytometry (FIG. 2E). Consistent with in vitro data, both endogenous and adoptively transferred tumor infiltrating macrophages exhibited elevated (approximately 200-fold increase by qPCR) ARG1 levels compared to liver-homing and resident macrophages respectively, confirming that adoptively transferred macrophages can alter their metabolic state in response to pathology present in the host. The tumor volumes of 25-50 mm³ also indicated that macrophages are present early in tumorigenesis and are a promising candidate for early cancer detection.

### Adoptively Transferred Macrophages Migrate to and Accumulate in Tumor Microenvironments

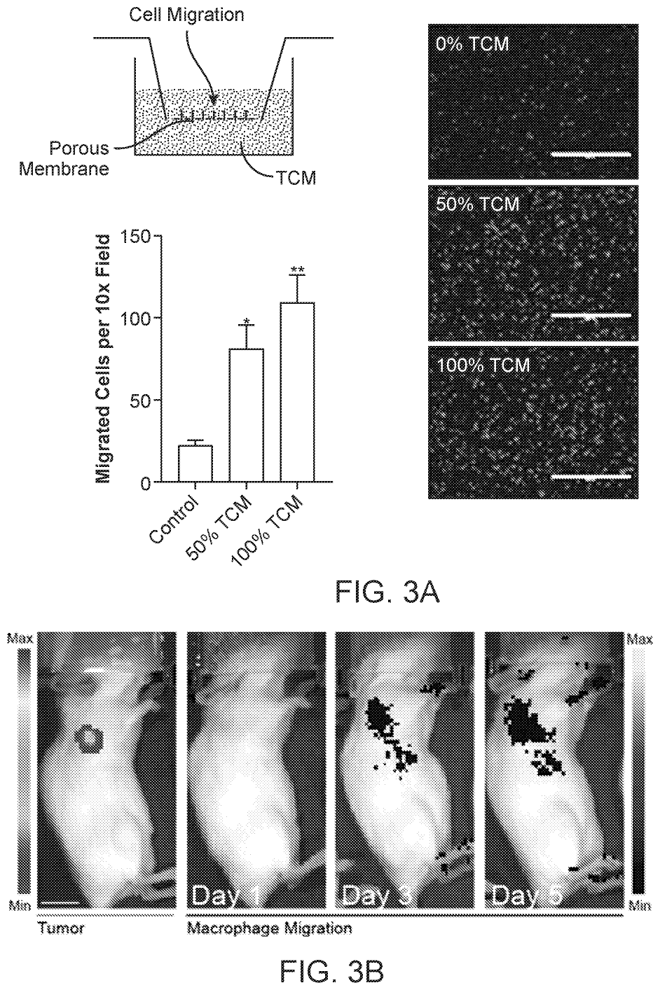

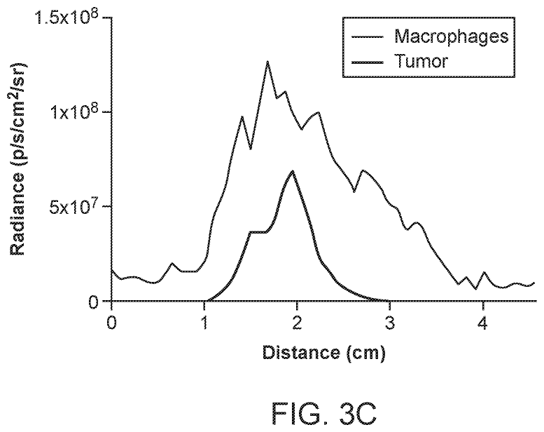

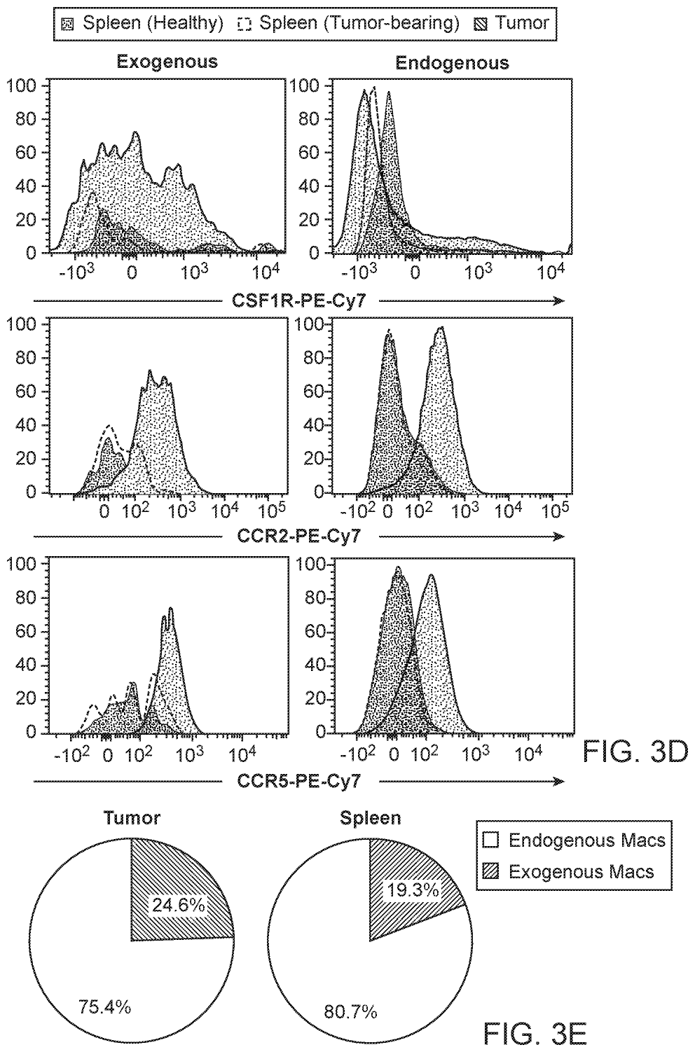

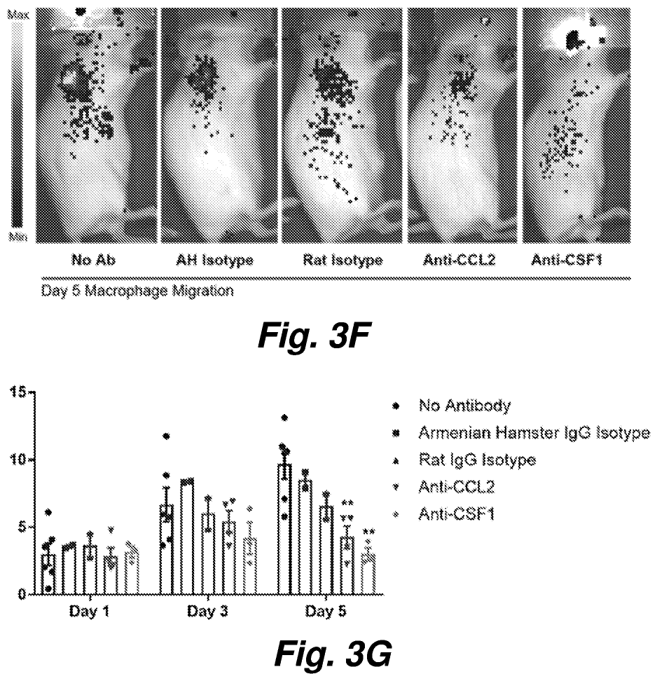

One barrier to translation of probe-based diagnostics is inefficient delivery to sites of disease. Since immune cell recruitment is a common feature of many disease states, including cancer, it was considered that a macrophage sensor would naturally migrate to sites of malignancy. Consistent with reports of macrophage chemotaxis towards tumor-derived cytokines such as CSF-1, macrophages exhibited concentration-dependent migration towards TCM up to 4-fold greater than toward unconditioned media in 24-hour transwell assays (FIG. 3A). In vivo, VT680 labeled adoptively transferred macrophages migrated to 25-50 mm³ subcutaneous colorectal tumors over the course of five days as visualized by fluorescence imaging of the near-infrared dye (FIGS. 3B-3C). Immunofluorescence of resected tumors revealed co-localization of macrophages with regions of hypoxia (FIG. 15). Fluorescence signal strongly co-localized with bioluminescence signal from Fluc transfected CT26 tumors as evidenced by radiance line-traces along the right shoulder (FIG. 3D). Flow cytometry analysis of both splenic and tumor macrophages revealed that 20-25% of macrophages present in each site were from adoptive transfer (CD11b+, F4/80+, VT680+), suggesting significant colonization of the sensor in both healthy organs and sites of disease (FIGS. 2D and 3E).

To investigate the mechanism of recruitment, surface expression of chemokine receptors described as playing a role in migration such as CSF1, CCL2, and CCL5 was analyzed. Both endogenous and adoptively transferred tumor-infiltrating macrophages exhibited increased expression of the cognate receptors CSF1 R, CCR2, and CCR5 relative to splenic macrophages (FIG. 3D). To delineate the role of chemokines in mediating either recruitment to or maintenance in the tumor, neutralizing doses of antibody against CCL2 and CSF1 were administered and again the migration of VT680+macrophages was monitored. Neutralization of either chemokine significantly diminished migration compared and confirmed the mechanism of recruitment (FIG. 3D).

### Secreted Biomarkers Enable Non-Invasive Monitoring of Macrophage Activation

To non-invasively assay changes in macrophage polarization in vivo, macrophages were engineered such that activation of ARG1 was coupled to production of a secreted biomarker that can be assayed in the blood or a peripheral blood sample from, a living subject. Gluc is a primarily (95%) secreted synthetic biomarker exhibiting high sensitivity relative to firefly and Renilla luciferases, is stable in serum with a 20-min half-life enabling dynamic monitoring of activation processes, and also offers the potential for spatial tracking of macrophage ARG1 expression by BLI of intracellularly trapped Gluc. Accordingly, the approximately 3.8 kb ARG1 promoter and enhancer region upstream of a secreted Gluc was cloned and engineered into a RAW264.7 cell line with stable expression of the pARG1-Gluc reporter construct for non-invasive monitoring of M2 polarization.

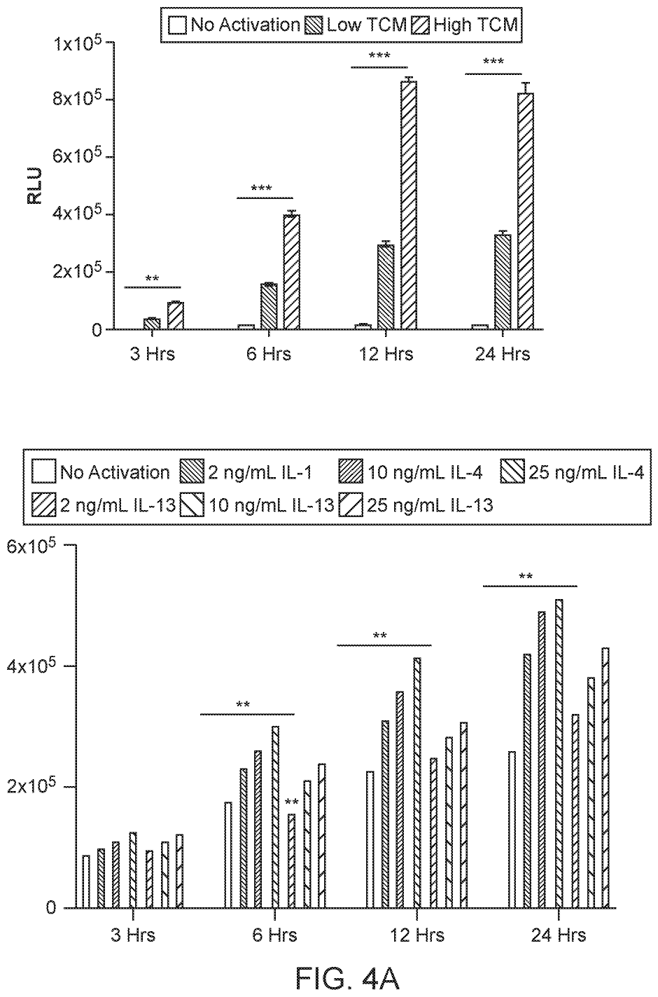

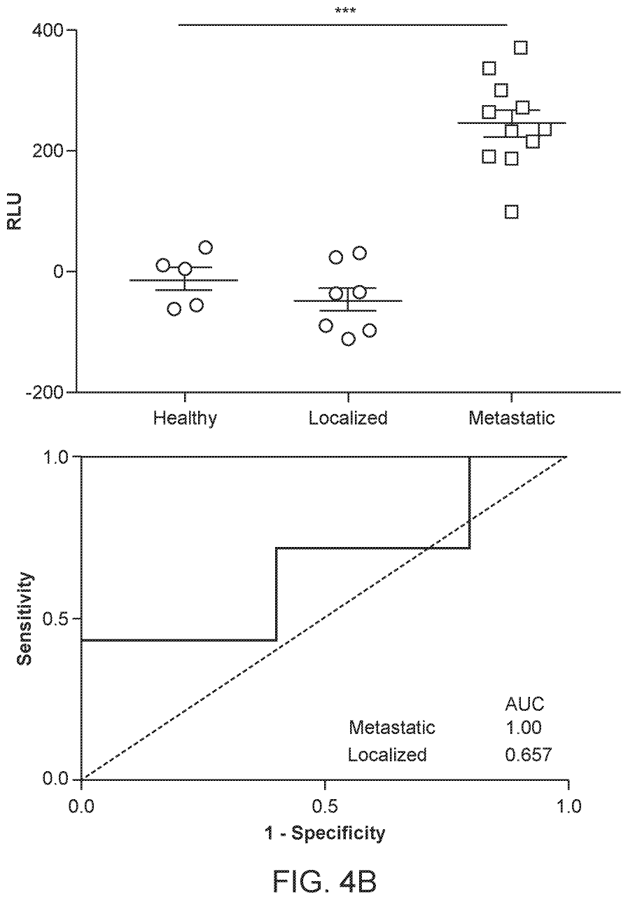

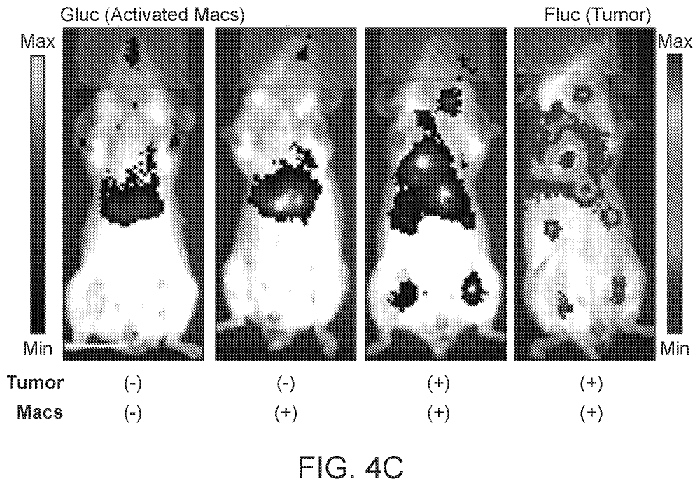

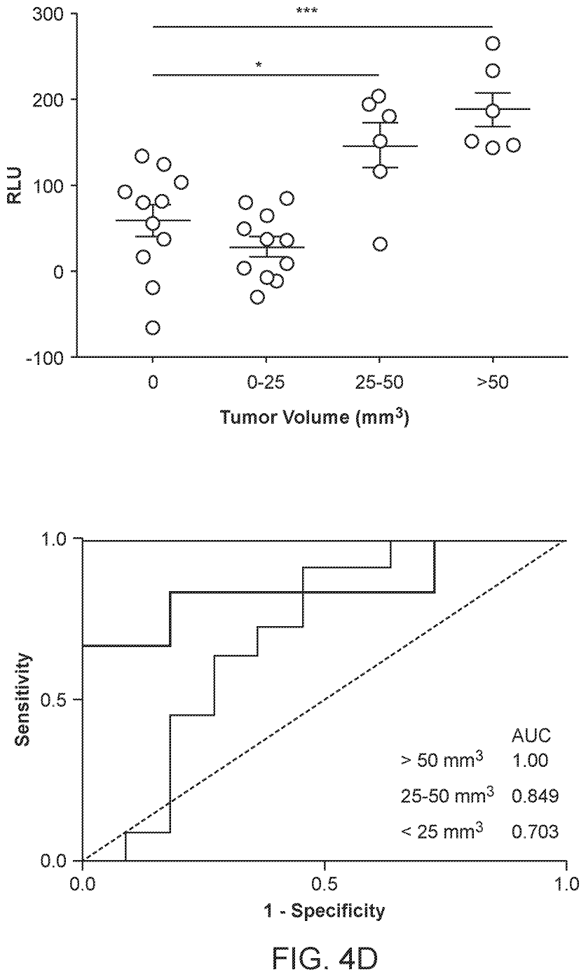

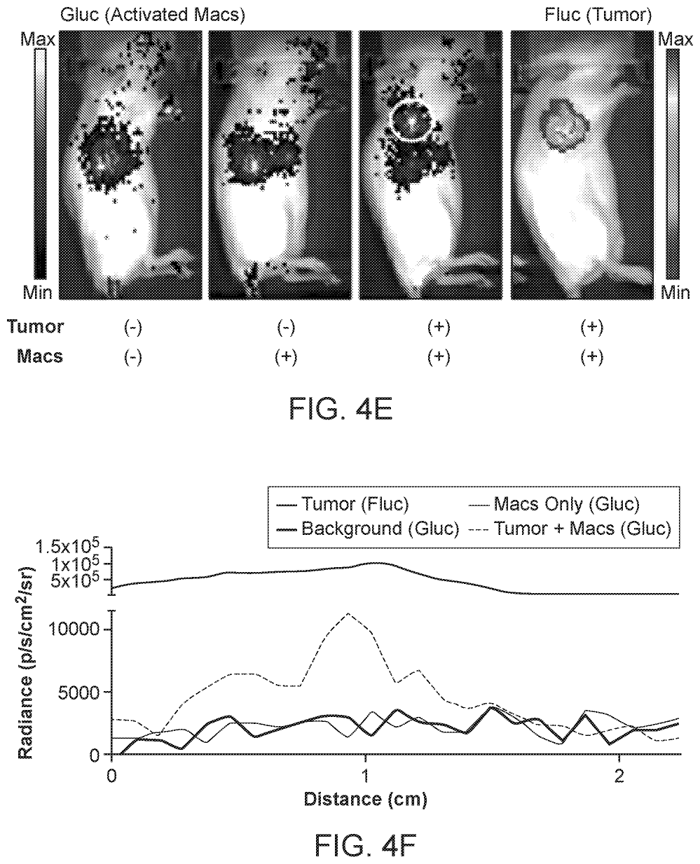

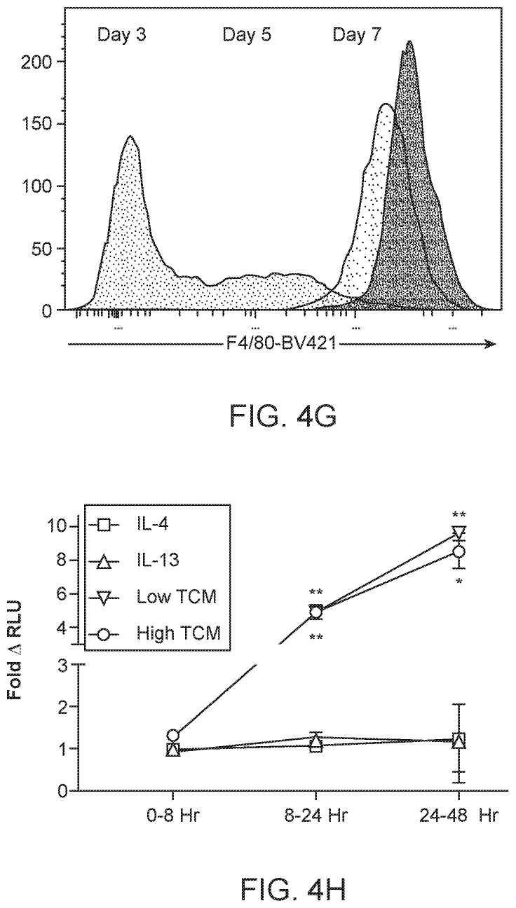

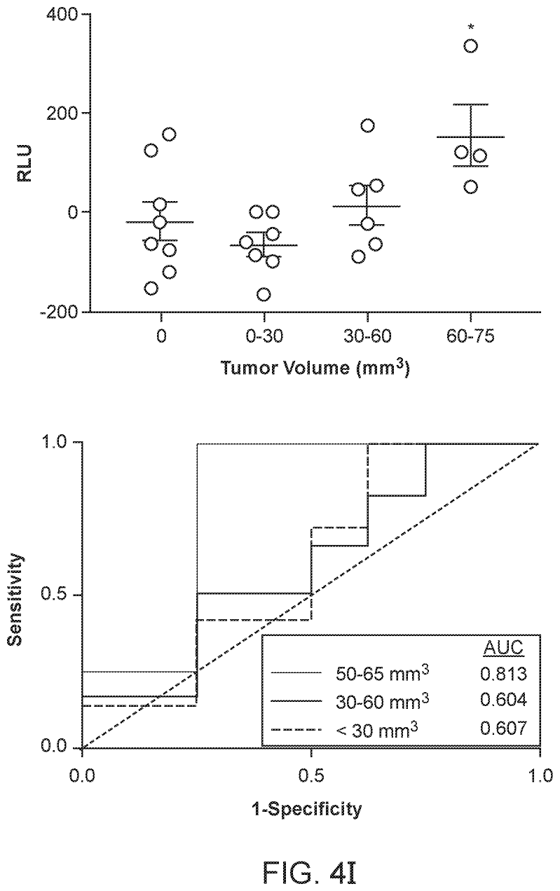

In ex vivo activation time course experiments with TCM, IL-4, and IL-13, sampling of culture media revealed a concentration and time dependent increase in Gluc luminescence signal of up to 2-fold for IL-4/IL-13 and 60-fold for “high” TCM over 24 hours (FIG. 4A).

### An Engineered Macrophage Sensor can Detect Sub-50 mm³ Tumors

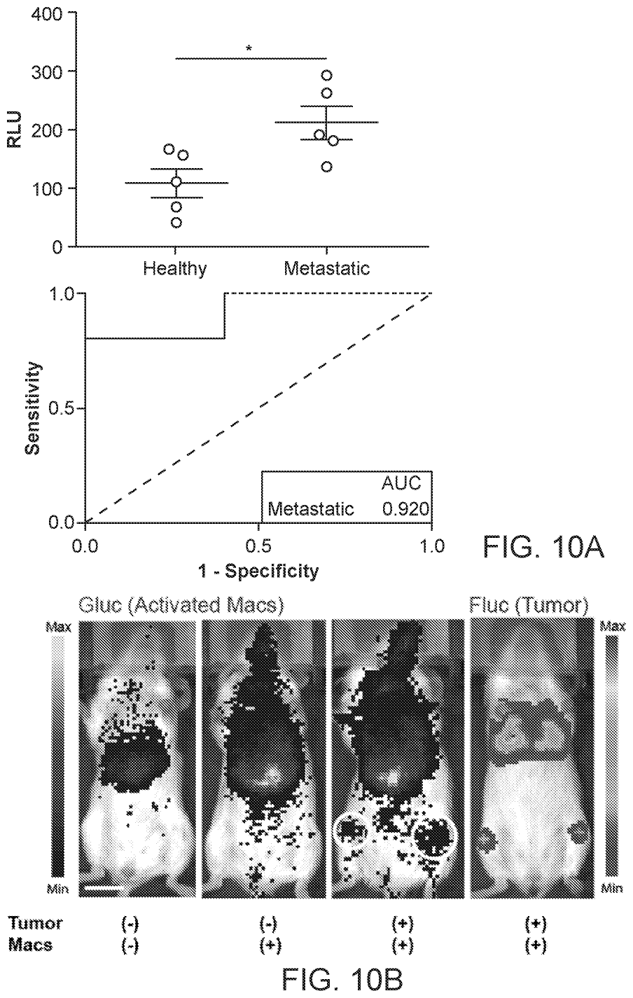

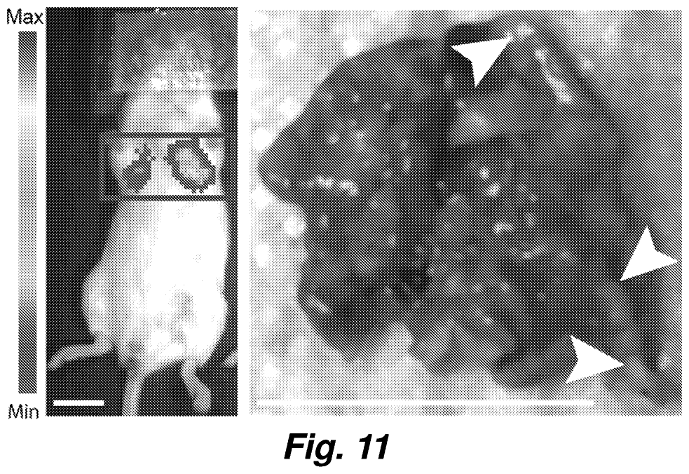

To determine whether the engineered macrophage platform could detect tumors in vivo, macrophages as described above containing the ARG-1 based sensor were introduced intravenously into tumor bearing mice and the plasma of the mice was subsequently assayed for Gluc to monitor activation. A syngeneic model of metastatic breast cancer was employed, wherein intravenously injected 4T1 cells first form localized microtumors in the lung followed by emergence of metastatic disease affecting the brain, liver, and bone. The sensor discriminated metastatic disease from healthy controls with 100% sensitivity and specificity (area under the curve (AUC) =1.00, n=11, p=0.0018) with plasma sampling 24 hours after injection (FIG. 4B). Similar results were achieved with transiently-transfected BMDMs, demonstrating the viability of the approach even with primary macrophages (FIG. 10A). This timescale is consistent with the kinetics of in vivo migration and macrophage activation in vitro. Furthermore, while Gluc is primarily a secreted biomarker, the -5% that remains concentrated intracellularly can be visualized with BLI to spatially track activated macrophages. Imaging of both the activated macrophages (Gluc) and metastases (Fluc) on separate days revealed marked co-localization of activated macrophages and sites of metastasis, including within the brain (FIG. 4C, FIG. 10B), indicating that the macrophage sensor effectively traffics to sites of disease and undergoes highly restricted patterns of activation. Notably, when the tumor burden was localized to non-palpable lung microtumors early after 4T1 injection (FIG. 4B, FIG. 11), the sensor did not detect disease possibly due to a poorly developed TME in the highly oxygenated lung.

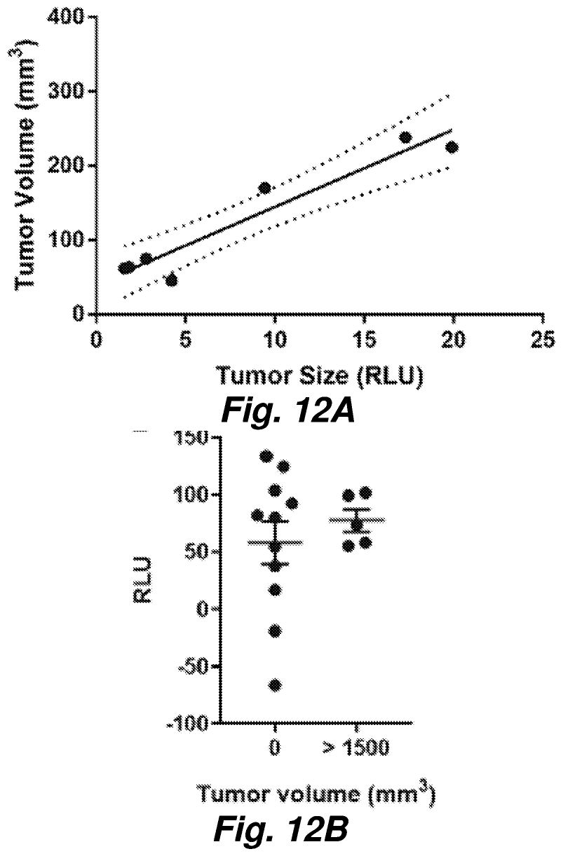

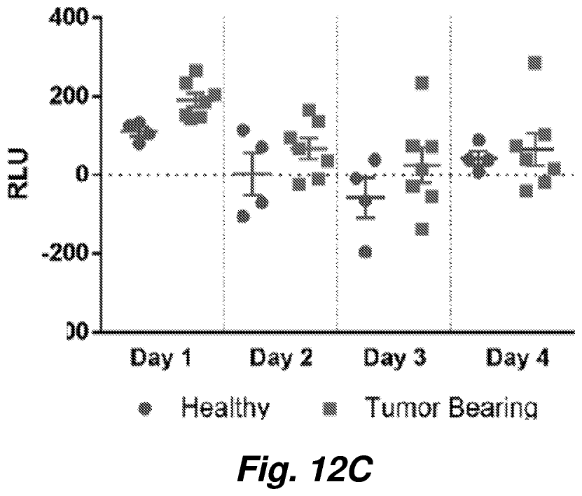

To determine the magnitude of tumor volumes that detectable with the sensor, the macrophage sensor described above was applied to a subcutaneous model of CT26 colorectal cancer with volumes of 0-250 mm³. Clinical PET can reliably detect tumor nodules of approximately 7 mm in diameter and volumes of approximately 200 mm³. Tumor size was measured with a caliper (FIGS. 12A and 12B) on the day of sensor injection and plasma was assayed on subsequent days. Caliper measurements correlated with tumor sizes measured by BLI (r²=0.918, FIG. 12A). At 24 hrs post-sensor injection, tumors with volumes greater than 50 mm³ (e.g. 50-250 mm³ average 117.19 +/−74.87 mm³) could be detected with 100% sensitivity and specificity (AUC=1.00, n=6, p=0.0009) (FIG. 3D). Notably, tumors with volumes of 25-50 mm³ (average 39.43 +/−7.90) were also discriminated from healthy controls with an AUC=0.849 (95% CI 0.620-1.00, n=6, p=0.021), suggesting that the activatable immune sensor described herein achieves a lower limit of detection than currently possible with clinical PET.

Tumor volumes of less than 25 mm³ were not reliably detected. Visibly necrotic or ulcerated tumors with volumes greater than about1500 mm³ were also not detected with the sensor (FIG. 12B), possibly due to limited immune infiltration into poorly vascularized necrotic cores even in the presence of a TME. While imaging of Gluc yielded non-specific liver signal from CTZ substrate metabolism, co-localization of activated macrophages with the site of the tumor was observed with BLI (FIG. 4E) and confirmed by radiance line-tracing across the right shoulder (FIG. 4F).

This localized model of disease also allowed interrogation of features of activation that would otherwise be difficult to study in a metastatic model that involves the added variable of cancer dissemination. For example, assaying of plasma for Gluc up to four days following sensor injection revealed a consistent decline in signal after 24 hours in both healthy and tumor bearing mice (FIG. 12C), potentially due to the immunogenicity and accelerated clearance of the synthetic biomarker after the first day. These early optimizations in a controlled model of localized disease argue for early plasma sampling and provide a framework for further cell number dose optimization in future studies.

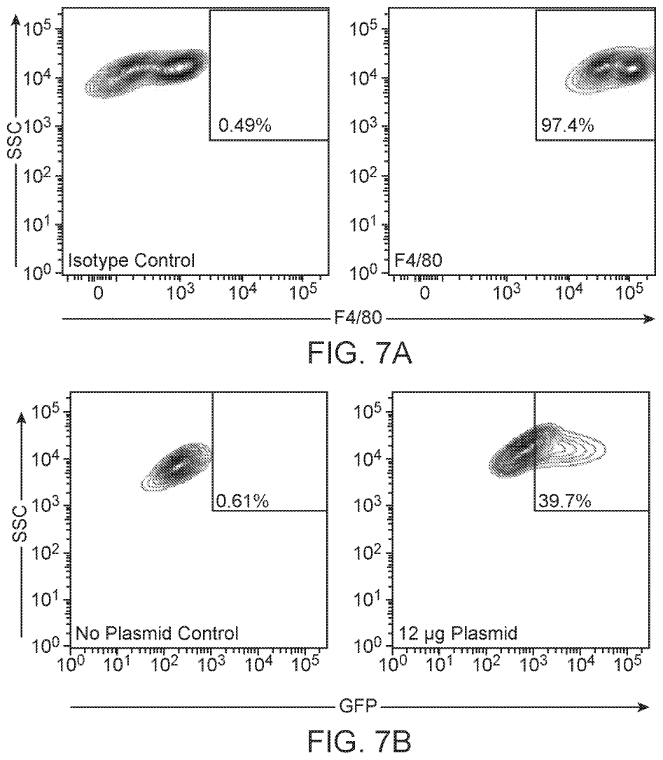

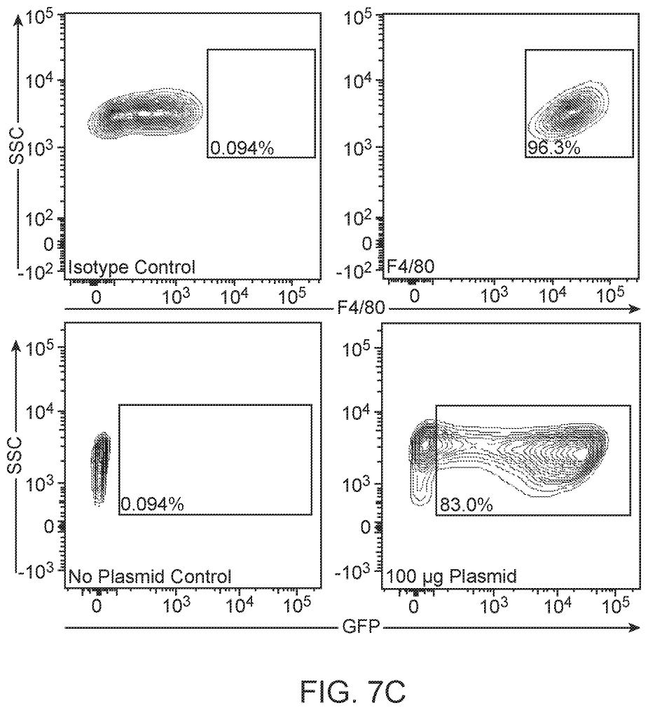

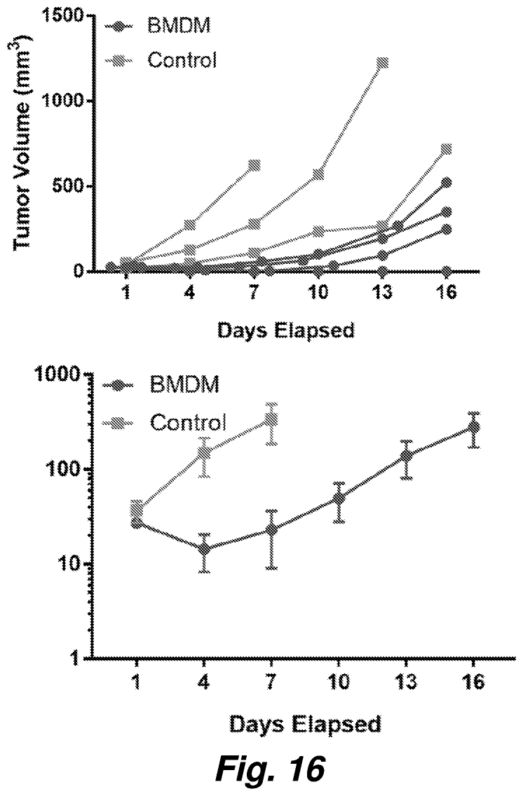

As further evidence for the translatability of the approach, tumor detection with primary BMDMs was also demonstrated. Monocytes were generated from bone marrow and phenotype confirmed by assaying for intermediate expression of the maturation marker F4/80 (FIG. 4G). The pARG1-Gluc construct was introduced by electroporation with efficiencies greater than 80% (FIG. 7B) and the resulting sensor was activated approximatelyl0-fold by both “low” and “high” TCM (FIG. 4H). The BMDM sensor also detected CT26 tumor volumes as low as 60-75 mm³ in vivo (n=4, p=0.0342) with an AUC of 0.813 (95% CI 0.555-1.00, n=4, p=0.0894) (FIG. 4I). Tumors from mice injected with BMDMs exhibited an initial regression (Day 4, p=0.058) but did not exhibit altered growth kinetics thereafter (FIG. 16) suggesting that sensor M2 polarization does not appear to accelerate tumor progression.

### Macrophage Sensor in a Model of Inflammation and Wound Healing

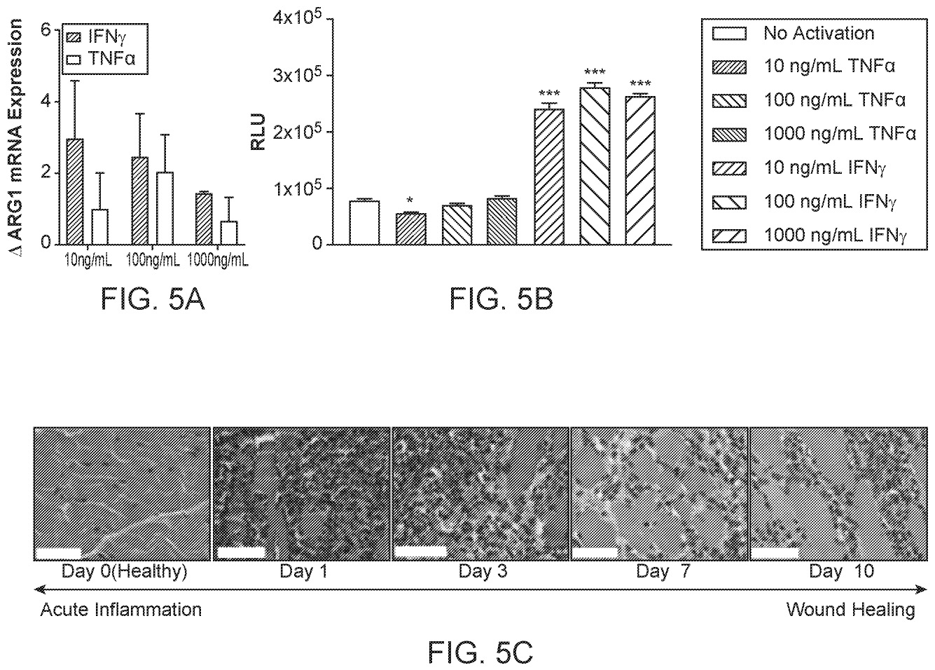

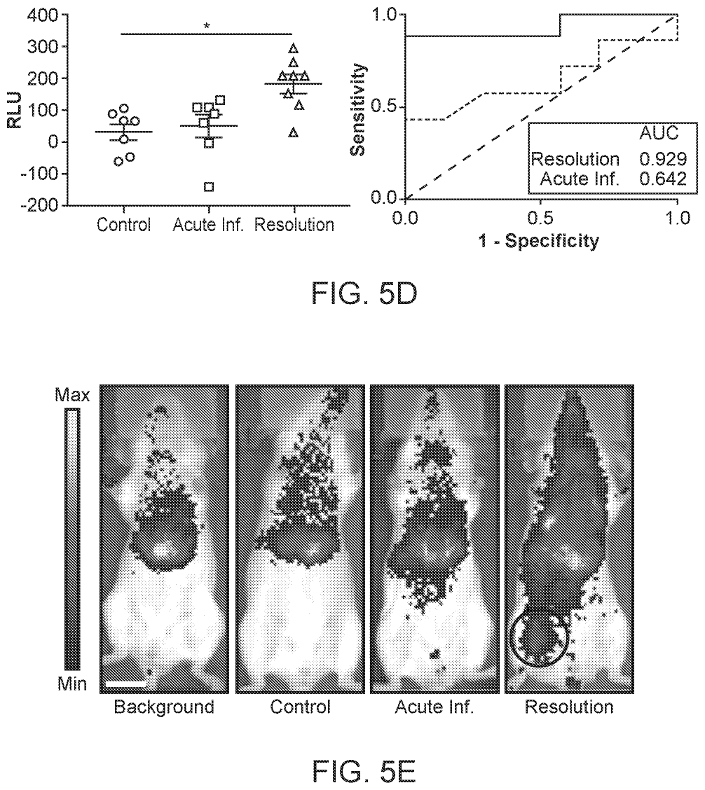

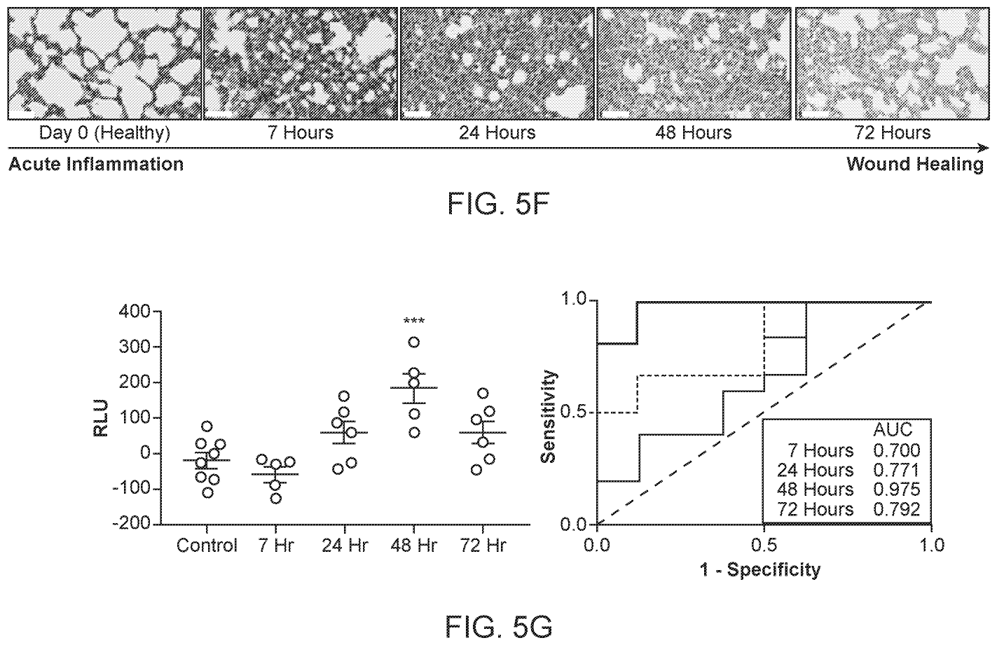

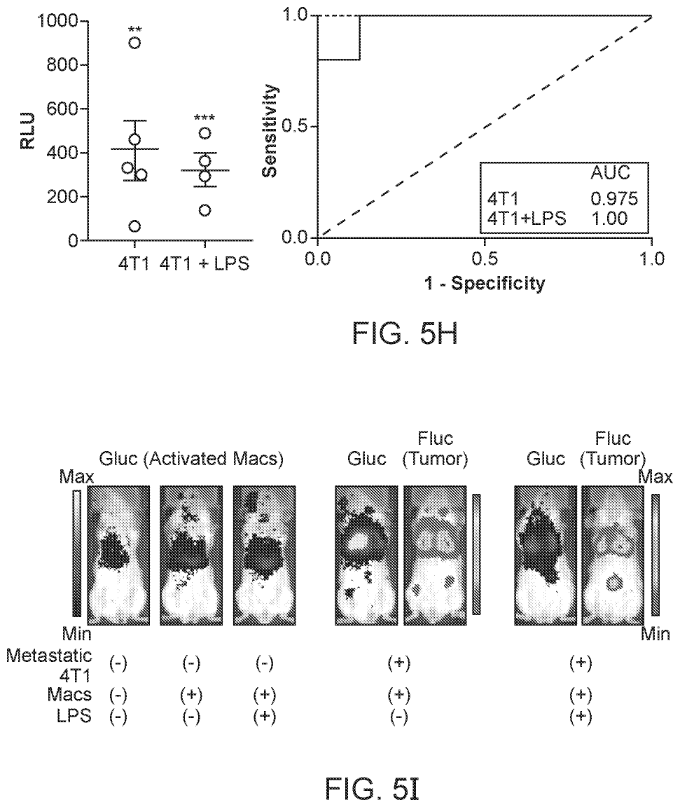

Inflammation is a significant confounding disease state for cancer diagnostics. The degree of false-positive sensor activation upon exposure to pro-inflammatory cytokines in vitro as well as in an in vivo model of inflammation was thus investigated. While immune infiltration is a hallmark of many pathologies, one advantage of using metabolic markers as a diagnostic surrogate is their tightly controlled and distinct transcriptional regulation in different disease states. Consistent with the well-characterized profile of pro-inflammatory M1 macrophages, RAW264.7 macrophages exhibited comparatively minimal elevations (less than 3-fold) or repression of ARG1 expression by qPCR upon stimulation with inflammatory cytokines such as IFNγ/LPS and TNFα (FIG. 5A). RAW264.7 ARG1 expression was similarly unaffected by TNFα, but was induced by IFNγ/LPS. This induction is mediated largely by LPS since high doses of IFNγ or TNFα did not significantly affect ARG1 expression. Stimulation of the pARG1-Gluc engineered BMDMs and RAW264.7 macrophages with these same inflammatory mediators led to minimal increases in Gluc secretion (FIG. 5B).

Sensor specificity was also evaluated in a model of turpentine oil-induced hind leg inflammation. Histology of hind leg muscle between one and ten days following intramuscular injection of turpentine oil revealed a stereotypical timeline of inflammation and wound healing: days 1-3 reflected an acute inflammatory phase characterized by profound neutrophil infiltration, while days 7-10 reflected a greater infiltration of debris-clearing macrophages and resolution of inflammation (FIG. 5C). Intravenous administration of the macrophage sensor of the disclosure on day 1 (during acute inflammation and on the same day as the turpentine oil) did not yield significantly elevated plasma Gluc after 24 hrs, corroborating the specificity of the immune sensor (FIG. 5D).

Further, since M2 macrophages are also involved in wound healing and resolution of inflammation, injection of the sensor on day 7 during the resolution phase was tested to determine if there would be sensor activation at the site of injection. Consistent with described biology of M2 activation and ARG1 induction during wound healing processes, plasma Gluc was significantly elevated when the sensor was injected during this phase (AUC=0.929, 95% CI 0.783-1.00, n=8, p=0.006). These temporal trends in activation were also apparent in BLI of Gluc taken 24 hours post-sensor injection (FIG. 5E).

Similar trends were observed using the BMDM sensor in an LPS-induced model of lung inflammation. Histology confirmed the kinetics of this model, revealing acute inflammation and an influx of neutrophils at 7 hours, wound healing and an influx of macrophages peaking at 48 hours, and a gradual restoration of healthy alveolar morphology at 72 hours (FIG. 5F). Intranasal administration of LPS either 7, 24, 48, or 72 hours prior to plasma sampling recapitulated these kinetics. Plasma Gluc was not elevated during acute inflammation at the 7 hour time point, providing further evidence for sensor specificity. As expected, plasma Gluc exhibited a gradual elevation during wound healing at 24 hrs (AUC=0.771, 95% CI 0.501-1.00, n=6, p=0.093) and eventually peaked at 48 hours (AUC=0.975, 95% CI 0.900-1.00, n=5, p=0.0054) (FIG. 5G). Levels began to decrease towards baseline by 72 hours (AUC=0.792, 95% CI 0.540-1.00, n=6, p=0.071) as normal lung architecture was restored.

The ability of co-occurring inflammation to affect the sensor's ability to detect tumors in vivo was also investigated. Employing the model of metastatic 4T1, no significant differences in plasma Gluc either in the absence (AUC=0.975, 95% CI 0.900-1.00, n=5, p=0.0054) or presence (AUC=1.00, 95% CI 1.00-1.00, n=4, p=0.0066) of LPS-induced acute lung inflammation was observed when using BMDM sensors (FIG. 5H). BLI of activated BMDM sensor was similarly unaffected (FIG. 5I).

While imaging of macrophages during acute inflammation did not reveal any activation or regional biasing, macrophage polarization in the right hind leg was easily observable during the resolution phase. The data indicate that while the immune sensor described herein can circumvent the most confounding disease state in cancer (acute inflammation), there are a cohort of pathologies (e.g. wound healing) that would be contraindicated for early cancer detection use. Alternatively, the robustness of macrophage phenotypic shifting argues for expanded uses of immune sensors outside of tumor detection, including applications in monitoring wound healing. The specific application will also influence the choice of promoter, with the selection of promoters other than pARG1 potentially allowing even greater specificity for early cancer detection.

Macrophage Sensor Outperforms a Clinically used Biomarker of Tumor Recurrence

Lastly, as evidence for the clinical relevance of an immune cell sensor, the ability of the macrophage sensor to detect the presence of a tumor earlier than a clinically used biomarker of recurrence/occurrence was evaluated. The sensor sensitivity was compared to carcinoembryonic antigen (CEA), a clinical biomarker used for therapy monitoring and detecting disease recurrence in colorectal carcinoma, since immune sensors would likely first be translated clinically for these indications. A subcutaneous model of colorectal adenocarcinoma using the CEA-secreting human cell line LS174T (which sheds CEA) in a subcutaneous localization in BALB/c NU/NU mice (n=7), was developed to account for the apparent lack of a CEA-secreting natural murine cell line. Once tumors reached an average volume greater 25 mm³, tumor size was measured by caliper and plasma was collected to check if CEA was detectable by enzyme linked immunosorbent assay (ELISA). The macrophage sensor was injected 24 hours prior to this day and Gluc levels were also measured from the same plasma sample. The plasma for CEA was then monitored every third day thereafter.

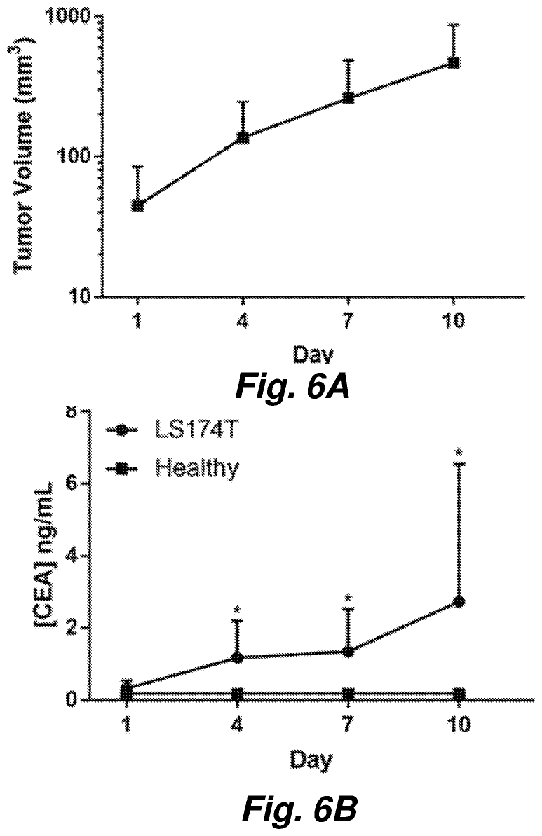

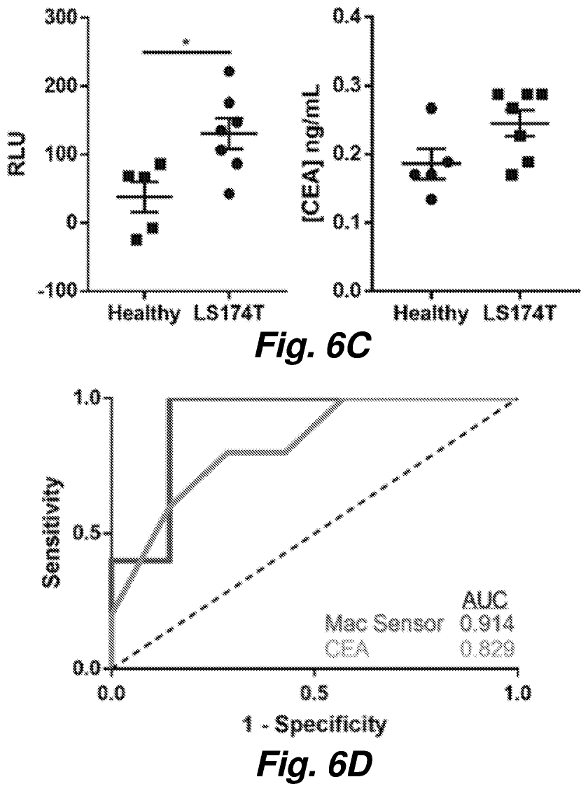

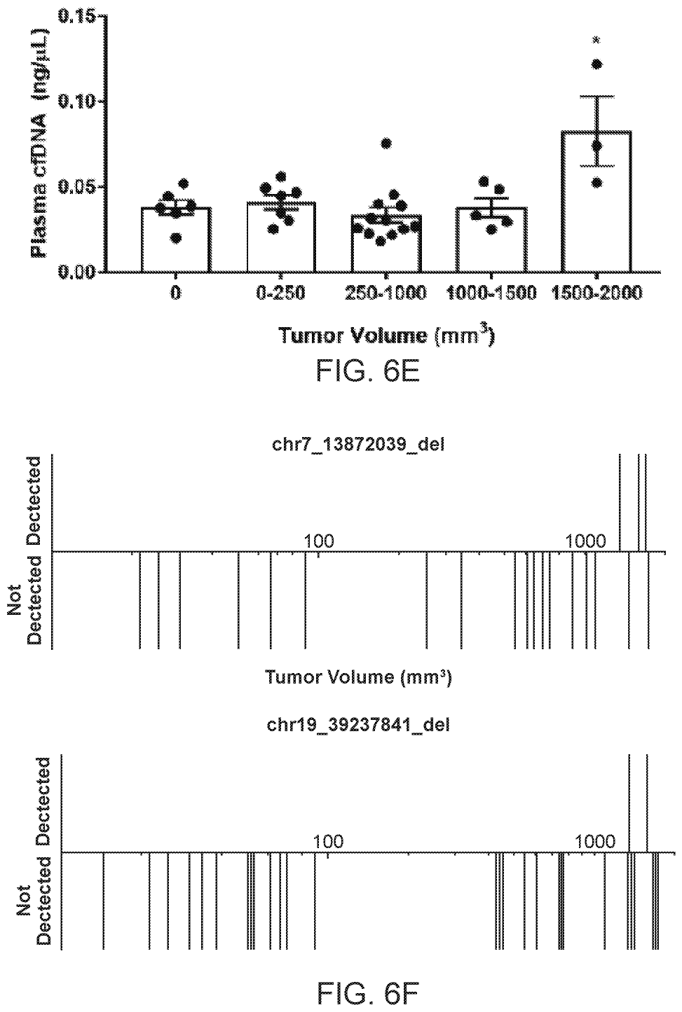

Tumor growth followed exponential kinetics with CEA being detectable no earlier than the second day of plasma sampling (Day 4) when tumors were of average volume 136.62 +/−110.71 mm³ (FIGS. 6A and 6B). During the first plasma sampling, tumors of average volume 44.82 +/−40.12 mm³ (67% smaller than on Day 4) were not detectable with statistical significance using CEA but were discriminated on the basis of Gluc measurements from the macrophage sensor (FIG. 6C). This is also reflected in the improved AUC from 0.829 (95% CI 0.590-1, p=0.062) with CEA to 0.914 (95% CI 0.738-1.00, p=0.019) with the macrophage sensor (FIG. 6D). The sensor's ability to outperform a clinically used biomarker is particularly promising given the inherent limitations of the model. For one, the NU/NU model lacks the endogenous immune cell cues that would otherwise contribute to mediating macrophage migration to a tumor and formation of a TME. In addition, LS174T is the second highest CEA-expressing cell line as characterized by ATCC, and it is likely that the gains in early detection by a macrophage sensor would be even more pronounced in tumor models that shed biomarkers less aggressively.

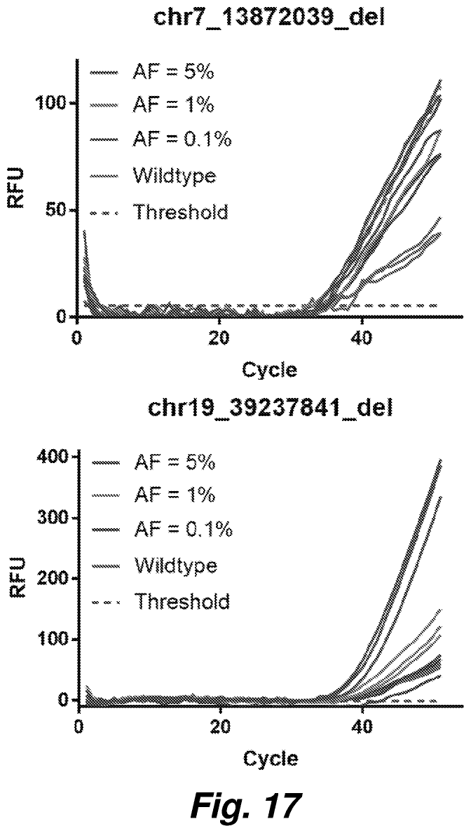

The sensitivity of the engineered immune cell sensor described herein was then compared to that of a second diagnostic modality, cell-free DNA (cfDNA). Since the sensor described herein can detect 25-50 mm³ CT26 tumors (FIG. 4D), the smallest size tumors that can be detected by either quantitation of cfDNA concentration or detection of mutations in the plasma was determined. Using a database of mutations in the CT26 cell line, two deletions were identified and qPCR assays were designed with allele frequency limits of detection of 0.1% and 1% respectively, which are similar to sensitivities of existing sequencing methods (FIG. 17).

cf DNA concentration was unable to discriminate healthy from tumor bearing mice until tumors reached volumes of 1500-2000 mm³ (FIG. 6E). Similarly, neither deletion mutation was detectable in the plasma until tumors had reached a minimum volume of 1300 mm³ (FIG. 6F). While the generalizability of our model is limited by variables such as tumor vascularization, rate of cell death, and kinetics of tumor DNA release, the data indicates that the macrophage sensor described herein can potentially detect tumors an order of magnitude smaller than possible with cfDNA even given a priori knowledge of the mutations.

### Kit

The disclosure also contemplates kits comprising one or more of compounds of the disclosure. In aspects of the disclosure, a kit of the disclosure comprises a container. In particular aspects, a kit of the disclosure comprises a container and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disease disclosed herein). A medicament or formulation in a kit of the disclosure may comprise any of the formulations or compositions disclosed herein.

One aspect of the disclosure encompasses embodiments of a genetically modified immune cell comprising a heterologous nucleic acid that expresses a detectable agent in response to a metabolic change induced by a pathological condition in an animal or human subject receiving the genetically modified immune cell.

In some embodiments of this aspect of the disclosure, the immune cell can be a monocyte, a macrophage, a T-cell, a B-cell, a natural killer cell, or a dendritic cell.

In some embodiments of this aspect of the disclosure, the heterologous nucleic acid can comprise at least one gene expression cassette comprising a gene expression regulatory region operably linked to a nucleic acid sequence encoding a detectable agent, and wherein the gene expression regulatory region can be responsive to a metabolic change in the genetically modified immune cell to induce expression of the detectable agent.

In some embodiments of this aspect of the disclosure, the heterologous nucleic acid can be a nucleic acid vector.

In some embodiments of this aspect of the disclosure, the heterologous nucleic acid can be a plasmid.

In some embodiments of this aspect of the disclosure, the gene expression regulatory region can comprise a gene promoter region.

In some embodiments of this aspect of the disclosure, the gene expression regulatory region can further comprise a gene-specific enhancer.

In some embodiments of this aspect of the disclosure, the gene promoter can be an ARG1 promoter, a CD163 promoter, a MMR/CD206 promoter, a CD200R promoter, a TGM2 promoter, a DecoyR promoter, an IL-1 R II promoter, an IL-10 promoter, a TGF-beta promoter, an IL-1ra promoter, a CCL17 promoter, a CCL2 promoter, or a CCL24 promoter.

In some embodiments of this aspect of the disclosure, the detectable agent can be a polypeptide or a secretable nucleic acid.

In some embodiments of this aspect of the disclosure, the detectable polypeptide can be Gaussia luciferase (Gluc).

In some embodiments of this aspect of the disclosure, the detectable polypeptide can be ferritin.

In some embodiments of this aspect of the disclosure, the detectable polypeptide can be HSV-1 thymidine kinase (HSV1-tk).

In some embodiments of this aspect of the disclosure, the detectable polypeptide can be a D8ORA mutant of the dopamine D2 receptor.

In some embodiments of this aspect of the disclosure, the detectable polypeptide can be a human sodium iodide symporter (hNIS).

In some embodiments of this aspect of the disclosure, the detectable polypeptide can be a contrast agent, a binding agent complementary to a reporter gene, an enzyme producing a detectable molecule, or a transporter driving accumulation of a detectable molecule.

In some embodiments of this aspect of the disclosure, the detectable agent can be a secretable nucleic acid.

In some embodiments of this aspect of the disclosure, the heterologous nucleic acid can have the nucleotide sequence as shown in SEQ ID NO: 1.

In some embodiments of this aspect of the disclosure, the gene expression regulatory region can be responsive to a tumor-specific metabolic change in the genetically modified immune cell to induce expression of the detectable agent.

In some embodiments of this aspect of the disclosure, the tumor-specific metabolic change in the genetically modified immune cell is induced by a cancer selected from the group consisting of: bladder cancer, breast cancer, colorectal cancer, endometrial cancer, head and neck cancer, lung cancer, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, liver cancer, medulloblastoma, neuroblastoma, brain tumor, osteosarcoma, malignant fibrous histiocytoma of bone, retinoblastoma, rhabdomyosarcoma, sarcoma, supratentorial primitive neuroectodermal tumor, pineal tumors, visual pathway and hypothalamic glioma, Wilms' tumor, esophageal cancer, hairy cell leukemia, kidney cancer, oral cancer, pancreatic cancer, skin cancer and small-cell lung cancer.

In some embodiments of this aspect of the disclosure, the pathology-specific metabolic change in the genetically modified immune cell can be from an inflammation.

Another aspect of the disclosure encompasses embodiments of a method of generating a genetically modified immune cell comprising the steps of: isolating from a human or animal subject a population of pathology-responsive immune cells; and transforming the isolated pathology-responsive immune cell population with a heterologous nucleic acid that expresses a detectable agent in response to a metabolic change induced by a pathological condition in an animal or human subject receiving the genetically modified immune cell.

In some embodiments of this aspect of the disclosure, the pathology can be a tumor.

In some embodiments of this aspect of the disclosure, the tumor-responsive immune cells are macrophages.

Yet another aspect of the disclosure encompasses embodiments of a method of detecting a pathological condition in an animal or human subject comprising the steps of: administering to an animal or human subject a pharmaceutically acceptable composition comprising a population of genetically-modified immune cells according to the disclosure; obtaining a biofluid sample from the animal or human subject; detecting in the biofluid sample the presence of the secretable detectable agent expressed by the genetically-modified immune cells in contact with or in the proximity of a pathological condition of the animal or human subject, wherein the presence indicates that the animal or human has a pathological condition inducing a metabolic change.

In some embodiments of this aspect of the disclosure, the biofluid can be blood.

In some embodiments of this aspect of the disclosure, the genetically-modified immune cells can be tumor-responsive macrophages.

In some embodiments of this aspect of the disclosure, the pathological condition can be a cancer.

In some embodiments of this aspect of the disclosure, the pathological condition can be a tumor.

In some embodiments of this aspect of the disclosure, the method further comprises the step of detecting a signal from the detectable agent within immune cells adjacent to or attaching to the pathological condition; generating an image of the detectable signal relative to the animal or human; and determining the position of the localized signal in the animal or human.

In some embodiments, the method can comprise detecting the absence of secretion and using such absence to assign the absence of pathology to the animal or human subject.

Still another aspect of the disclosure encompasses embodiments of a kit comprising an apparatus for bone marrow derived macrophage (BMDM) isolation; and an endotoxin-free preparation of a plasmid encoding a detectable agent operably linked to an ARG-1 promoter.

Another aspect of the disclosure encompasses embodiments of a method for identifying a pathological condition in a subject, comprising: (a) administering to the subject a genetically modified immune cell comprising a heterologous nucleic acid having a nucleic acid sequence that encodes a detectable agent, wherein the genetically modified immune cell expresses the detectable agent in response to a metabolic change induced by a pathological condition in the subject, and (b) detecting the detectable agent in the subject to identify the pathological condition.

In some embodiments of this aspect of the disclosure, when responsive to a tumor-specific metabolic change in the genetically modified immune cell, the gene expression regulatory region can induce expression of the detectable agent.

In embodiments of this aspect of the disclosure, the tumor-specific metabolic change in the genetically modified immune cell can be induced by a cancer selected from the group consisting of: bladder cancer, breast cancer, colorectal cancer, endometrial cancer, head and neck cancer, lung cancer, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, liver cancer, medulloblastoma, neuroblastoma, brain tumor, osteosarcoma, malignant fibrous histiocytoma of bone, retinoblastoma, rhabdomyosarcoma, sarcoma, supratentorial primitive neuroectodermal tumor, pineal tumors, visual pathway and hypothalamic glioma, Wilms' tumor, esophageal cancer, hairy cell leukemia, kidney cancer, oral cancer, pancreatic cancer, skin cancer and small-cell lung cancer.

In some embodiments of this aspect of the disclosure, the pathology-specific metabolic change in the genetically modified immune cell is from an inflammation.

In some embodiments of the genetically modified immune cell the heterologous nucleic acid comprises a plurality of different gene expression regulatory regions wherein each regulatory region is operably linked to a plurality of nucleic acid sequence encoding a multiple types of detectable agent, and wherein the gene expression regulatory region is responsive to a pathology-specific metabolic change to induce expression of the detectable agent, of which the levels of each detectable agent are indicative of a different condition of the subject.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

Tumor infiltrating leukocyte profiling: The fractional immune cell makeup from transcriptomic profiling of various cancers was obtained from the Stanford University iPRECOG database. As multiple cohorts of tumor samples are listed under each cancer type, a weighted average of the immune cell fractions was calculated for each cancer based on the number of tumor samples analyzed in each cohort.

### Example 2

Bone marrow-derived macrophage (BMDM) preparation and electroporation: Femurs and tibias from 6-8 week old female BALB/c mice were isolated and bone marrow flushed with 5 mL cold PBS. Marrow was re-suspended into a homogenous solution by repeated pipetting and passed through a 40 μm filter to eliminate debris. After centrifugation for 5 min at 300×g, marrow was re-suspended in ACK lysis buffer (Invitrogen, Waltham, Mass.) for 5 min on ice. ACK was diluted 10-fold in PBS, and the solution centrifuged again for 5 min at 300 ×g. Cells were plated at a density of 4×10⁶cells/10 cm petri dish in 10 mL of IMDM (ThermoFisher, Waltham, Mass.) supplemented with 10% heat-inactivated fetal bovine serum (FBS), 1% antibacterial/antimycotic (A/A) solution (ThermoFisher), and 10 ng/mL murine colony stimulating factor (M-CSF, Peprotech, Rocky Hill, N.J.) and maintained in a humidified, 5% CO₂ incubator at 37° C. for 5 days prior to harvesting with a cell lifter for downstream use.

To make BMD monocytes for all in vivo studies, cells were plated at a density of 6×10⁶ cells/well in 6-well Corning® Costar® Ultra-Low Attachment Plates (Corning, Corning, N.Y.) in 6 mL of media supplemented with 20 ng/mL M-CSF. After 5 days, adherent cells (macrophages) were discarded and the non-adhered cell were collected. Purity was determined to be greater than 96% by flow cytometry via staining for F4/80 (Biolegend, San Diego, Calif.) compared to an isotype control (FIG. 7A).

Transient transfection was performed by electroporation using a Nucleofector kit for mouse macrophages (Lonza, Basel, Switzerland) and protocol Y-001 on the associated Nucleofector 2b Device. Each reaction contained 2×10⁶ BMDMs and 12 μg plasmid DNA and achieved on average approximately 40% efficiency (FIG. 7B).

### Example 3

Cell Lines: RAW264.7 murine macrophage, CT26 murine colon carcinoma, 4T1 murine breast cancer, and LS174T human colorectal adenocarcinoma were obtained from ATCC (Manassas, Va.) and cultured in either DMEM (RAW264.7, LS174T) or RPMI (CT26, 4T1) supplemented with 10% FBS and 1% antibacterial/antimycotic solution (ThermoFisher) and maintained in a humidified, 5% CO₂ incubator at 37° C. CT26 eGFP-firefly luciferase (Fluc) and 4T1 eGFP-Fluc cell lines were generated by lentiviral transduction followed by three rounds of sorting for the highest 2.5% of eGFP expressers. The RAW264.7 arginase-1 promoter driving Gaussia luciferase (pARG1-Gluc) cell line was generated by transfection with Lipofectamine 3000 (ThermoFisher) and three rounds of sorting for the highest 2.5% of eGFP expressers.

### Example 4

In vitro macrophage activation: Macrophages (RAW264.7 or BMDM) were plated at a density of 1×10⁶ cells/well in 6-well plates in 2.5 mL of medium. After 24 hrs, media was either replaced with tumor conditioned media (TCM), or was supplemented with IL-4, IL-13, tumor necrosis factor alpha (TNFα), or interferon gamma (IFNγ). “High” and “low” TCM were generated by culturing 2×10⁶ or 3×10⁶ CT26 cells, respectively, in 2.5 mL media per well in a 6-well plate for 24 hrs. Conditioned media was centrifuged for 10 min at 300 ×g to eliminate debris prior to use. After 24 hrs, macrophages were either harvested for RNA isolation or 20 μL of culture media was collected to assay for Gluc using a BioLux Gaussia Luciferase Assay Kit (New England BioLabs, Ipswich, Mass.) according to manufacturer's instructions. Luminescence measurements were performed on a TD 20/20 luminometer (Turner Designs, San Jose, Calif.) with 10 seconds of integration and luminescence expressed in relative light units (RLU).

### Example 5

Arginase (ARG-1) gene expression assays: Total RNA was extracted from macrophages using the RNeasy Mini Kit (Qiagen, Hilden, Germany) following the manufacturer's instructions. Extraction of RNA from macrophages in cell culture was by direct lysis within the well, while extraction from tumor- and spleen-infiltrating macrophages was performed by direct sorting into RNeasy lysis buffer during flow cytometry. cDNA synthesis used the iScript cDNA synthesis kit (Bio-rad, Hercules, Calif.) following the manufacturer's instructions. Quantitative PCR (qPCR) reactions were in 20 μL volumes containing 1× SsoAdvanced Universal Probes Supermix (Bio-Rad), 1 μL of gene-specific hydrolysis probe, 2 μL of cDNA, and nuclease-free water (Bio-rad). FAM fluorophore-conjugated hydrolysis probes for ARG1 and GAPDH were commercially obtained (Bio-rad). Thermal cycling for both cDNA synthesis and qPCR used a CFX96 Real-Time System C1000 Touch Thermal Cycler (Bio-Rad) using the following protocols: 25° C. for 5 min, 46° C. for 20 min, 95° C. for 1 min (cDNA synthesis) and 95° C. for 3 min, followed by 60 cycles of: 95° C. for 15 seconds and 59° C. for 30 secs (qPCR). Technical replicates for all samples were performed in duplicate. Negative controls were performed with nuclease-free water instead of cDNA. The cycle threshold was a single threshold determined automatically (using the CFX Manager Software Version 3.1) with all C_{q} values falling within the linear quantifiable range of the assay.

### Example 6

Arginase (ARG-1) activity assay: Macrophages were washed once with PBS, harvested, and lysed in 100 μL Pierce IP Lysis Buffer (ThermFisher) containing 1× Halt Protease Inhibitor Cocktail (ThermoFisher) for 10 min on ice. Lysate was centrifuged at 4° C. for 10 min at 14,000 ×g and supernatant arginase activity was measured using the colorimetric QuantiChrom Arginase Assay Kit (BioAssay Systems, Hayward, Calif.) following manufacturer instructions. Optical density at 430 nm was measured on a Synergy 4 microplate reader (BioTek, Winooski, Vt.).

### Example 7

In vitro migration assay: In vitro migration assays were performed using 6.5 mm Transwell tissue culture-treated inserts with 8.0 μM pore size polyester membranes (Corning, Corning, N.Y.). 1×10⁵ RAW264.7 macrophages were seeded in 100 μL of DMEM on the top of the membrane chamber and allowed to adhere for 10 min prior to submerging of the chamber into wells containing either “high” tumor conditioned or unconditioned media. After 24 hrs, the insert was removed, non-migrated cells on the top of the membrane were removed with a cotton swab, and the insert was fixed in 600 μL of 70% ethanol for 10 min. Membrane was allowed to dry for 15 min and then submerged into 600 μL of 0.2% crystal violet (CV) solution for 10 min for cell staining. Finally, the membrane was washed 5 times with PBS to remove excess CV, removed from the insert, and number of migrated cells were counted in brightfield in 10 random 10x fields of view on an EVOS imaging system (ThermoFisher).

### Example 8

In vivo migration assay: Female BALB/c mice 6-8 week old (Charles River, Wilmington, Mass.) were subcutaneously implanted with 1×10⁶CT26 eGFP-Fluc cells in 100 μL PBS on the right shoulder. After seven days, tumors were imaged by bioluminescence imaging (BLI) on an IVIS Spectrum (PerkinElmer, Waltham, Mass.) device after intraperitoneal injection of 30 mg/kg D-Luciferin in 100 μL PBS to confirm tumor intake. Ten days after tumor implantation, 1×10⁷ syngeneic RAW264.7 macrophages were labeled with a near infrared fluorescent membrane dye (VivoTrack 680, PerkinElmer) and injected intravenously in 100 μL PBS. In vivo fluorescence imaging with the 640 nm excitation and 700 nm emission filter set was performed using the IVIS Spectrum on days one, three, and five after macrophage injection to visualize migration to the tumor. Regions of interest (ROIs) and line traces were drawn using Living Image 4.5.2 software to quantitate extent of macrophage migration and co-localization with tumor, respectively, based on average radiance in photons·s⁻¹·cm⁻²·steradian⁻¹.

### Example 9

Macrophage staining and cell sorting: Resected tumors were mechanically dissociated with scissors and digested in 5 mL Hank's Balanced Salt Solution (HBSS) containing 10 μg/mL DNase I (Sigma-Aldrich, St. Louis, Mo.) and 25 μg/mL Liberase (Roche, Basel, Switzerland) for 45 min at 37° C. The solution was then diluted with cold PBS, filtered through a 70 μm filter, and centrifuged for 5 min at 300 ×g prior to resuspension in FACS buffer (PBS +2% FBS +1% A/A). Harvested spleens were pressed through 40 μm filters using the back of a 1 mL syringe plunger and washed through with PBS. After spinning for 5 min at 300 ×g, splenocytes were re-suspended in 5 mL of ACK lysis buffer and put on ice for 5 min. The red blood cell free fraction was then centrifuged and re-suspended in FACS buffer.

For macrophage sorting experiments, single cell suspensions from tumor and spleen were stained in 100 μL HBSS containing 0.2 μg of each antibody against F4/80 and CD11 b (Biolegend) as well as a live/dead stain (propidium iodide) and sorted on a FACSAria II benchtop cell sorter (Becton Dickinson, Franklin Lakes, N.J.) with compensation performed using UltraComp eBeads (ThermoFisher) or single-stained cells for VivoTrack 680. Positive and negative cells were gated using fluorescence minus one controls.

### Example 10

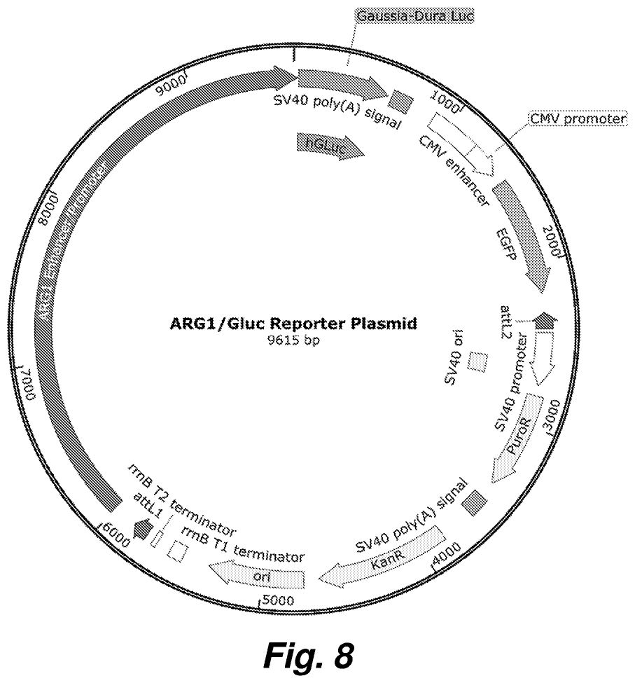

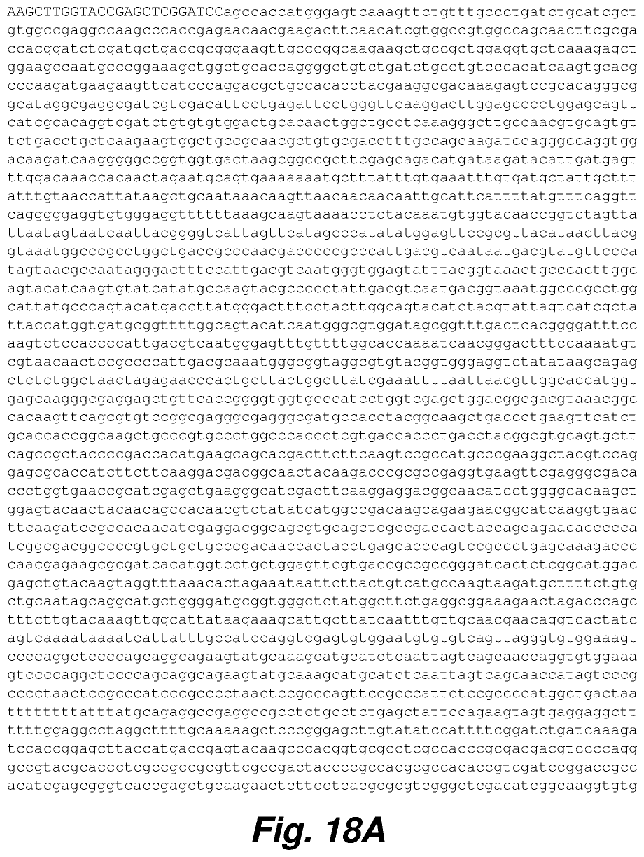

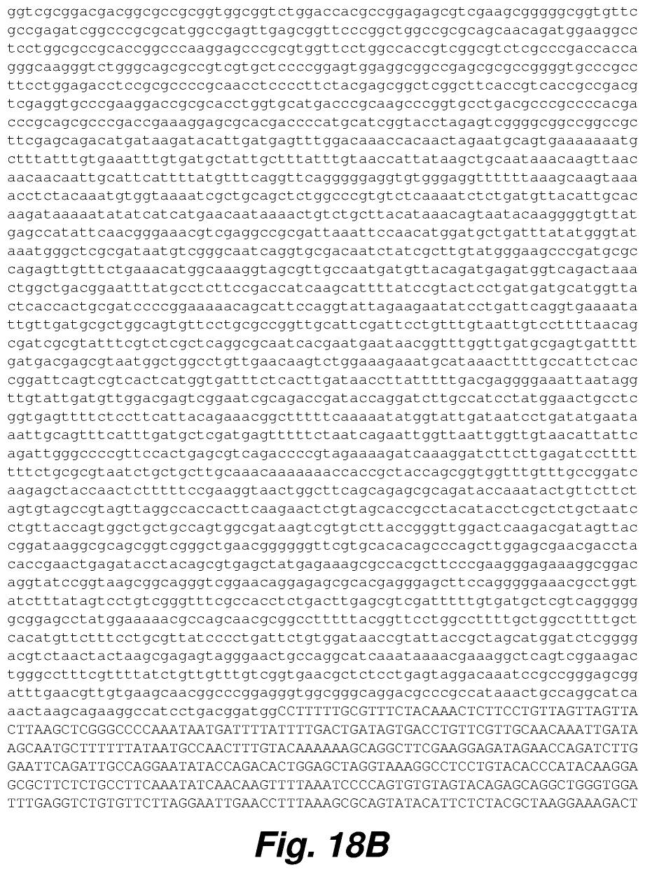

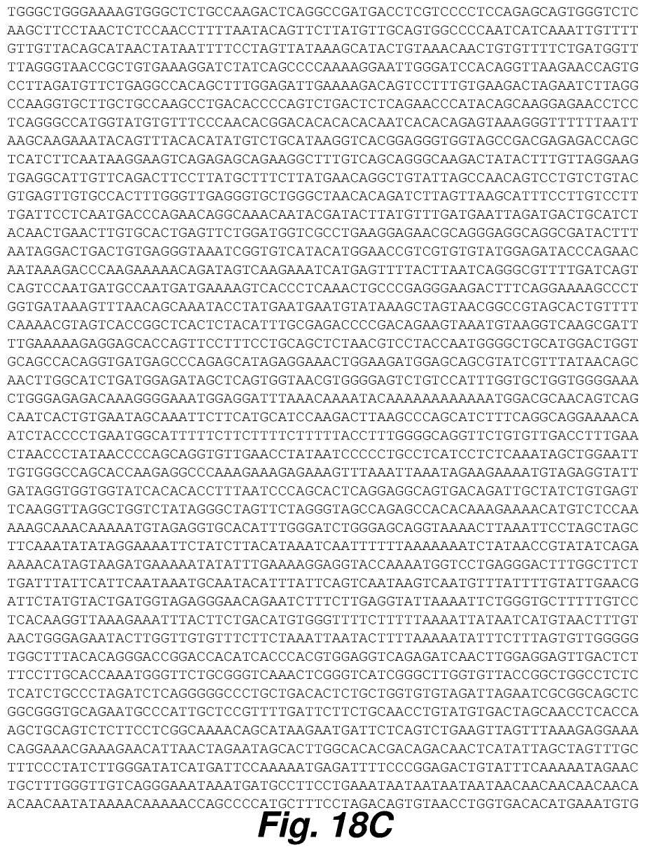

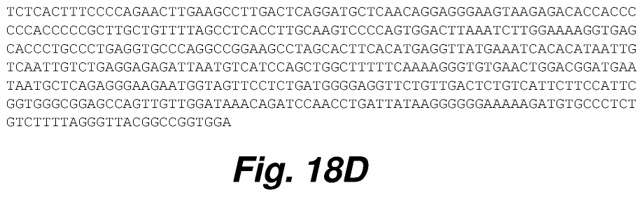

Reporter plasmid construction: Plasmids were constructed using standard PCR-based cloning techniques and sequenced by Sequetech (Mountain View, Calif.). The pARG1-Gluc reporter plasmid (FIG. 8) was formed by cloning the -31/-3810 ARG1 promoter/enhancer (Addgene, Cambridge, Mass.) sequence described previously upstream of the sequence for Gaussia Dura Luciferase (Genecopoeia, Rockville, Md.). The nucleotide sequence is given in SEQ ID NO: 1 as shown in FIG. 18.

### Example 11

Mouse tumor models and blood collection: A syngeneic model of metastatic breast cancer was established by intravenous injection of 2.5×10⁵ 4T1 eGFP-Fluc cells in 150 μL PBS into female BALB/c mice. In the localized model of disease, a macrophage sensor (3×10⁶ cells RAW264.7 or 1×10⁶ BMDM) was injected after seven days when disease was still likely restricted to the lungs as visualized by BLI. In the metastatic model, a sensor was injected after 14 days once tumor burden had spread beyond the lung. Mice were bled from the submandibular vein 24 hrs after sensor injection, blood was collected in K3-EDTA tubes (Greiner, Baden-Wurttemberg, Germany), and then centrifuged at 4° C. for 10 min at 1,000 ×g. Gluc was assayed from 20 μL plasma as described previously. Activated macrophages (intracellular Gluc) were imaged by BLI 48 hrs after macrophage injection by intravenous injection of 35 μg coelenterazine (CTZ) substrate (Promega, Madison, Wis.) diluted in 150 μL of PBS.

A syngeneic subcutaneous colorectal cancer model was established as described in the migration studies with tumors allowed to grow to either 0-25 mm³, 25-50 mm³, or 50-200 mm³ prior to engineered macrophage injection. Tumor volumes were approximated by the equation V=0.5×L×W² with L and W representing the longer and shorter immediately perpendicular diameters of the tumor spheroid. Dimensions were measured with a digital caliper. Mice were bled (50 μL) from the submandibular vein at 24-hour intervals for up to four days following sensor injection with BLI imaging of activated macrophages performed 48 hrs after injection.

### Example 12

Murine inflammation models: In the model of muscle inflammation, 6-8 week old female BALB/c mice were injected intramuscularly with 30 μL turpentine oil (Sigma-Aldrich) in the right hind limb. Healthy contralateral muscle injected with PBS and inflamed muscle from mice sacrificed on days one, three, seven, and ten after injection were collected and fixed in 10% formalin for 48 hrs, embedded in paraffin, and processed for Hematoxylin & Eosin (H&E) staining following standard protocols (Histo-Tec Laboratory, Hayward, Calif.). RAW264.7 macrophage sensor (3×10⁶ cells) was injected intravenously in 100 μL PBS on either day one or day seven after turpentine oil injection with 50 μL blood collection, plasma Gluc measurement, and BLI of activated macrophages occurring 24 hrs after cell injection.

In the LPS-induced model of acute lung inflammation, 6-8 week old female BALB/c mice were inoculated intranasally with 50 μg of LPS resuspended in 20 μL PBS. Control mice received no vehicle as intranasal administration of saline can induce lung inflammation. Healthy lungs or lungs at 7, 24, 48, 72 hrs after LPS administration were fixed and processed for H&E staining as previously described. In the monitoring of wound healing, BMDM sensor (3×10⁶live, transfected cells) was injected intravenously in 100 μL PBS and LPS was administered either 0, 24, 48, or 65 hrs after injection. Since plasma Gluc from the BMDM sensor was assayed 72 hrs after injection, this schedule allowed for interrogation of sensor activity at either 72, 48, 24, or 7 hrs of inflammation respectively.

In the model of co-occurring tumors and acute inflammation, Balb/c mice bearing metastatic 4T1 tumors were injected with BMDM sensor and inoculated intranasally with LPS 65 hrs afterwards (7 hrs prior to assaying plasma Gluc). BLI of activated BMDM sensor was performed immediately following blood collection.

### Example 13

Carcinoembryonic antigen release model: LS174T cells (2×10⁶ in 50 μL PBS) were implanted subcutaneously on the right shoulder of female immunodeficient BALB/c NU/NU mice (Charles River). Tumor volumes were approximated by caliper measurements and blood (50 μL) was collected every three days starting on the tenth day post-implantation. RAW264.7 macrophage sensor (3×10⁶ cells in 100 μL PBS) was injected on day 1 (10 days after implantation) with 50 μL blood collected 24 hrs afterwards for Gluc and CEA detection. Plasma CEA concentration was measured with a commercial ELISA kit (ThermoFisher) with detection limit of 200 pg/mL.

### Example 14

Statistical Analysis: Statistical analysis was performed using parametric unpaired t tests with Welch's correction. All statistical analysis was performed in GraphPad Prism version 7.03.

### Example 15

Immunofluorescence: 6-8 week old female BALB/c mice bearing 10-day old CT26 tumors were injected intravenously with 1×10⁷ BMDMs labeled with CellBrite™ Fix 640 dye (Biotium, Fremont, Calif.) according to manufacturer's instructions. After four days, tumors were harvested 90 minutes following intraperitoneal injection of 60 mg/kg pimonidazole hydrochloride (Hypoxyprobe, Burlington, Mass.) for detection of hypoxia. Tumors were frozen in optimal cutting temperature compound (Sakura Finetek, Torrance, Calif.) immediately following excision, cut into 10 μm-thick sections using a microtome, and mounted onto frosted microscope slides. Tissue slides were then blocked for 30 minutes with immunofluorescence blocking buffer (Cell Signaling Technology, Danvers, Mass.) prior to staining with 1:50 (1.2 μg/mL) FITC anti-pimonidazole (Hypoxyprobe) overnight at 4° C. Slides were washed three times with PBS and coverslips mounted using ProLong® Gold Antifade Reagent with DAPI (ThermoFisher) prior to sealing with clear nail polish. Images were acquired using a NanoZoomer 2.0-RS whole slide imager (Hamamatsu, Hamamatsu City, Japan).

### Example 16

Cell-free DNA model: Subcutaneous CT26 tumors were grown to volumes between 0-2000 mm³ and mice were terminally bled from the submandibular vein. Cell-free DNA (cfDNA) was extracted from the plasma using the NextPrep-Mag™ cfDNA Isolation Kit (Bioo Scientific, Austin, Tex.) and quantitated using the Quant-iT™ High-Sensitivity dsDNA Assay Kit (ThermoFisher). We confirmed that the cfDNA exhibited a primarily mononucleosome size profile (140-180 base pairs) using an Agilent 2100 Bioanalyzer (Agilent, Santa Clara, Calif.) and excluded samples with contamination of large fragment genomic DNA. Primer and locked nucleic acid (LNA) probes were obtained from IDT (San Jose, Calif.) with sequences shown in Table 1.

TABLE 1
Parameters for cell-free DNA
mutation detection assay

| Locus | chr7_13872039 |
|---|---|
| Deletion | CAGGCCAGTTTCATCCCTTC |
| | (SEQ ID NO: 2) |
| Forward Primer | ATTCCCAAAGCGTCGAACT |
| | (SEQ ID NO: 3) |
| Reverse Primer | CTACCATTGGAAGGACGATCAC |
| | (SEQ ID NO: 4) |
| LNA Probe | TAA + G + GA + CA + T + |
| | CC + AT (SEQ ID NO: 5) |
| Amplicon Size (bp) | 65 |
| Locus: | chr19_39237841 |
| Deletion: | G |
| Forward Primer: | CAATTCTTTAGGTGTACCCTGTG |
| | (SEQ ID NO: 6) |
| Reverse Primer: | AAACAATGGAGCAGATGACATT |
| | (SEQ ID NO: 7) |
| LNA Probe | TT + C + T + CG + C + |
| | T + GT (SEQ ID NO: 8) |
| Amplicon Size (bp): | 76 |

‘+’ after a base in the probe sequence indicates that the base is a locked nucleic acid base.
LNA, Locked Nucleic Acid.

Thermal cycling for qPCR were performed using a CFX96 Real-Time System C1000 Touch Thermal Cycler (Bio-Rad) using the following protocol: 95° C. for 3 min, followed by 50 cycles of 95° C. for 15 secs and 69.4° C. (chr7_13872039_del) or 67.9° C. (chr19_39237841_del) for 15 secs. Allele frequency limit of detection experiments were performed with genomic DNA isolated from the CT26 cells using the PureLink™ Genomic DNA Mini Kit (ThermoFisher) that was diluted with healthy Balb/c genomic DNA (Sigma-Aldrich) to obtain allele frequencies of 5%, 1%, and 0.1% based on initial allele frequencies of 100% (chr7_13872039_del) and 9% (chr19_39237841_del) previously reported. Reactions contained 5 ng of cfDNA, forward and reverse primer concentrations of 500 nM, and probe concentration of 200 nM in 20 μL volume total.

The disease-activatable probes of the disclosure are advantageous when the biological and mathematical limitations faced by endogenous biomarkers are considered. In cf DNA, for example, mutation allele frequency decreases with disease burden, leading to an increasing probability that there will not exist a single copy of a mutation in a 10 mL blood draw. It has been estimated that tumors must reach volumes greater than 1,000 mm³ (corresponding to allele frequencies of 0.01%) for there to exist even one genome equivalent of tumor DNA in 4 mL of plasma. The macrophage sensors of the disclosure, however, are able to detect tumors up to 50-fold smaller in volume than is possible with cf DNA mutation detection. Methods of biomarker detection using the macrophage sensors of the disclosure do not require knowing which DNA mutations to look for.

The approach described herein has the advantage of modularity in choice of disease, immune cell, and reporter. Other diseases with an immune component beyond cancer including, but not limited to, autoimmune diseases such as Hashimoto's thyroiditis, rheumatoid arthritis, diabetes mellitus type 1, and systemic lupus erythematosus or inflammatory diseases including, but not limited to, atherosclerosis, diabetes, pancreatitis, COPD, chronic kidney disease, acute kidney injury, ulcerative colitis (UC) and Crohn disease, non-alcoholic fatty liver disease, epilepsy, Alzheimer's and Parkinson's disease. Other immune subtypes beyond macrophages (e.g. T-cells, B-cells, and natural killer cells) all modulate metabolic genes in tumors and, therefore, can be advantageously detected using the engineered immune cells and methods of the disclosure. Further, while Gaussia luciferase (Gluc) is useful as a reporter, other non-immunogenic synthetic biomarkers such as secreted placental alkaline phosphatase (SEAP), human chorionic gonadotropin (HCG), synthetic RNA or synthetic miRNa templates, halves of a split reporter molecule may be used.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A genetically modified immune cell comprising a heterologous nucleic acid configured to express a detectable agent in response to a metabolic or molecular expression change induced by a pathological condition in an animal or human subject receiving the genetically modified immune cell.

2. The genetically modified immune cell of claim 1, wherein the genetically modified immune cell is a monocyte, a macrophage, a T-cell, a B-cell, a natural killer (NK) cell, a myeloid cell, a stem cell, or a dendritic cell.

3. The genetically modified immune cell of claim 1, wherein the heterologous nucleic acid comprises a gene expression regulatory region operably linked to a nucleic acid sequence encoding a detectable agent, and wherein the gene expression regulatory region is responsive to a pathology-specific metabolic change to induce expression of the detectable agent.

4. The genetically modified immune cell of claim 1, wherein the heterologous nucleic acid is a nucleic acid vector.

5. The genetically modified immune cell of claim 4, wherein the heterologous nucleic acid is a plasmid.

6. The genetically modified immune cell of claim 1, wherein the gene expression regulatory region comprises a gene promoter region.

7. The genetically modified immune cell of claim 6, wherein the gene expression regulatory region further comprises a gene-specific enhancer.

8. The genetically modified immune cell of claim 6, wherein the gene promoter is an ARG1 promoter, an AKT1 promoter, a versican promoter, a MIF promoter, a Ym1 promoter, a CD206 promoter, a FIZZ1 promoter, a DC-SIGN promoter, a CD209 promoter, a MGL-1 promoter, a Dectin -1 promoter, a CD23 promoter, a galectin-3 promoter, a Mer tyrosine kinase promoter, an AXL receptor protein promoter, a GAS-6 promoter, a NOS-2 promoter, a CD68 promoter, a CD86 promoter, a CCL18 promoter, a CD163 promoter, a MMR/CD206 promoter, a CD200R promoter, a TGM2 promoter, a DecoyR promoter, an IL-1 R II promoter, an IL-10 promoter, a TGF-beta promoter, an IL-1ra promoter, a CCL17 promoter, a CCL2 promoter, or a CCL24 promoter.

9. The genetically modified immune cell of claim 1, wherein the detectable agent is a detectable polypeptide or a secretable nucleic acid.

10. The genetically modified immune cell of claim 9, wherein the detectable polypeptide is a contrast agent, a binding agent complementary to a reporter gene, an enzyme producing a detectable molecule, a photoacoustic reporter, a bioluminescent reporter, an autofluorescent reporter, a chemiluminescent reporter, a luminescent reporter, or a colorimetric reporter, an agent that can be detected by non-invasive imaging, or a transporter driving accumulation of a detectable molecule.

11. The genetically modified immune cell of claim 9, wherein the detectable agent is a secretable nucleic acid, and wherein the secretable nucleic acid is a structured RNA or a synthetic miRNA detectable by RT-QPCR, QPCR, hybridization, sequencing, or mass spectroscopy.

12. The genetically modified immune cell of claim 10, wherein the detectable polypeptide is ferritin.

13. The genetically modified immune cell of claim 10, wherein the detectable polypeptide is a Gaussia luciferase (Gluc).

14. The genetically modified immune cell of claim 10, wherein the detectable polypeptide is HSV1-tk.

15. The genetically modified immune cell of claim 10, wherein the detectable polypeptide is a D8ORA mutant of the dopamine D2 receptor.

16. The genetically modified immune cell of claim 10, wherein the detectable polypeptide is a human sodium iodide symporter (hNIS).

17. The genetically modified immune cell of claim 1, wherein the heterologous nucleic acid has at least 80% identity to the nucleotide sequence as shown in SEQ ID NO: 1.

18. The genetically modified immune cell of claim 1, wherein the heterologous nucleic acid encodes the detectable agent.

19. The genetically modified immune cell of claim 3, wherein responsive to a tumor-specific metabolic change in the genetically modified immune cell, the gene expression regulatory region induces expression of the detectable agent.

20. The genetically modified immune cell of claim 19, wherein the tumor-specific metabolic change in the genetically modified immune cell is induced by a cancer selected from the group consisting of: bladder cancer, breast cancer, colorectal cancer, endometrial cancer, head and neck cancer, lung cancer, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, liver cancer, medulloblastoma, neuroblastoma, brain tumor, osteosarcoma, malignant fibrous histiocytoma of bone, retinoblastoma, rhabdomyosarcoma, sarcoma, supratentorial primitive neuroectodermal tumor, pineal tumors, visual pathway and hypothalamic glioma, Wilms' tumor, esophageal cancer, hairy cell leukemia, kidney cancer, oral cancer, pancreatic cancer, skin cancer and small-cell lung cancer.

21. The genetically modified immune cell of claim 3, wherein the pathology-specific metabolic change in the genetically modified immune cell is from an inflammation.

22. A method of generating a genetically modified immune cell comprising the steps of:
(a) isolating from a human or animal subject a population of pathology-responsive immune cells; and
(b) transforming a pathology-responsive immune cell of the isolated population of pathology-responsive immune cells isolated in (a) with a heterologous nucleic acid to yield the genetically modified immune cell, wherein the heterologous nucleic acid encodes a detectable agent, and wherein the genetically modified immune cell is configured to express the detectable agent in response to a metabolic change induced by a pathological condition in an animal or human subject receiving the genetically modified immune cell.

23. The method of claim 22, wherein the pathology-responsive immune cells are tumor-responsive immune cells.

24. The method of claim 23, wherein the tumor-responsive immune cells are macrophages.

25. A method of detecting a pathological condition in an animal or human subject comprising the steps of:
administering to a subject a pharmaceutically acceptable composition comprising a population of genetically-modified immune cells according to claim 1;
obtaining a biofluid sample from the animal or human subject;
detecting in the biofluid sample the presence of the secretable detectable agent wherein the presence indicates that the animal or human subject has a pathological condition inducing phenotypic change in the genetically-modified immune cells in contact with a pathological condition of the animal or human patient.

26. The method of claim 25, wherein the genetically-modified immune cells are tumor-responsive macrophages.

27. The method of claim 25, wherein the pathological condition is a cancer.

28. The method of claim 25, wherein the pathological condition is a tumor.

29. The method of claim 25, wherein the method further comprises the step of:
detecting a signal from the detectable agent in pathology-responsive immune cells adjacent to or attaching to the pathological condition;
generating an image of the detectable signal relative to the subject; and
determining the position of the localized signal in the subject.

30. The method of claim 25, wherein the biofluid is blood.

31. The method of claim 28, comprising performing the method when an amount of the detectable agent is not secreted by the genetically-modified immune cells adjacent to or attaching to a pathological condition of the animal or human patient.

32. A kit, comprising:
an apparatus for bone marrow derived macrophage (BMDM) isolation; and
an endotoxin-free preparation of a plasmid encoding a detectable agent operably linked to an Arginase-1 (Arg-1) promoter.

33. A method for identifying a pathological condition in a subject, comprising:
(a) administering to the subject a genetically modified immune cell comprising a heterologous nucleic acid having a nucleic acid sequence that encodes a detectable agent, wherein the genetically modified immune cell expresses the detectable agent in response to a metabolic change induced by a pathological condition in the subject, and
(b) detecting the detectable agent in the subject to identify the pathological condition.

34. The method of claim 33, wherein when responsive to a tumor-specific metabolic change in the genetically modified immune cell, the gene expression regulatory region induces expression of the detectable agent.

35. The method of claim 33, wherein the tumor-specific metabolic change in the genetically modified immune cell is induced by a cancer selected from the group consisting of:
bladder cancer, breast cancer, colorectal cancer, endometrial cancer, head and neck cancer, lung cancer, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease leukemia, liver cancer, medulloblastoma, neuroblastoma, brain tumor, osteosarcoma, malignant fibrous histiocytoma of bone, retinoblastoma, rhabdomyosarcoma, sarcoma, supratentorial primitive neuroectodermal tumor, pineal tumors, visual pathway and hypothalamic glioma, Wilms' tumor, esophageal cancer, hairy cell leukemia, kidney cancer, oral cancer, pancreatic cancer, skin cancer and small-cell lung cancer.

36. The method of claim 33, wherein the pathology-specific metabolic change in the genetically modified immune cell is from an inflammation.

37. The genetically modified immune cell of claim 1, wherein the heterologous nucleic acid comprises a plurality of different gene expression regulatory regions wherein each regulatory region is operably linked to a plurality of nucleic acid sequence encoding a multiple types of detectable agent, and wherein the gene expression regulatory region is responsive to a pathology-specific metabolic change to induce expression of the detectable agent, of which the levels of each detectable agent are indicative of a different condition of the subject.


SEQUENCE LISTING (8 sequences)

SEQ ID NO: 1 - DNA, 9615 bp, Artificial sequence
  Plasmid ARG1/Gluc Reporter Plamid
aagcttggtaccgagctcggatccagccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagctggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccctggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacaaccggtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattttaattaacgttggcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggtttaaacactagaaataattcttactgtcatgccaagtaagatgcttttctgtgctgcaatagcaggcatgctggggatgcggtgggctctatggcttctgaggcggaaagaactagacccagctttcttgtacaaagttggcattataagaaagcattgcttatcaatttgttgcaacgaacaggtcactatcagtcaaaataaaatcattatttgccatccaggtcgagtgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaaagatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgctagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccagatcttggaattcagattgccaggaatataccagacactggagctaggtaaaggcctcctgtacacccatacaaggagcgcttctctgccttcaaatatcaacaagttttaaatccccagtgtgtagtacagagcaggctgggtggatttgaggtctgtgttcttaggaattgaacctttaaagcgcagtatacattctctacgctaaggaaagacttgggctgggaaaagtgggctctgccaagactcaggccgatgacctcgtcccctccagagcagtgggtctcaagcttcctaactctccaaccttttaatacagttcttatgttgcagtggccccaatcatcaaattgttttgttgttacagcataactataattttcctagttataaagcatactgtaaacaactgtgttttctgatggttttagggtaaccgctgtgaaaggatctatcagccccaaaaggaattgggatccacaggttaagaaccagtgccttagatgttctgaggccacagctttggagattgaaaagacagtcctttgtgaagactagaatcttaggccaaggtgcttgctgccaagcctgacaccccagtctgactctcagaacccatacagcaaggagaacctcctcagggccatggtatgtgtttcccaacacggacacacacacaatcacacagagtaaagggttttttaattaagcaagaaatacagtttacacatatgtctgcataaggtcacggagggtggtagccgacgagagaccagctcatcttcaataaggaagtcagagagcagaaggctttgtcagcagggcaagactatactttgttaggaagtgaggcattgttcagacttccttatgctttcttatgaacaggctgtattagccaacagtcctgtctgtacgtgagttgtgccactttgggttgagggtgctgggctaacacagatcttagttaagcatttccttgtcctttgattcctcaatgacccagaacaggcaaacaatacgatacttatgtttgatgaattagatgactgcatctacaactgaacttgtgcactgagttctggatggtcgcctgaaggagaacgcagggaggcaggcgatactttaataggactgactgtgagggtaaatcggtgtcatacatggaaccgtcgtgtgtatggagatacccagaacaataaagacccaagaaaaacagatagtcaagaaatcatgagttttacttaatcagggcgttttgatcagtcagtccaatgatgccaatgatgaaaagtcaccctcaaactgcccgagggaagactttcaggaaaagccctggtgataaagtttaacagcaaatacctatgaatgaatgtataaagctagtaacggccgtagcactgttttcaaaacgtagtcaccggctcactctacatttgcgagaccccgacagaagtaaatgtaaggtcaagcgattttgaaaaagaggagcaccagttcctttcctgcagctctaacgtcctaccaatggggctgcatggactggtgcagccacaggtgatgagcccagagcatagaggaaactggaagatggagcagcgtatcgtttataacagcaacttggcatctgatggagatagctcagtggtaacgtggggagtctgtccatttggtgctggtggggaaactgggagagacaaaggggaaatggaggatttaaacaaaatacaaaaaaaaaaaatggacgcaacagtcagcaatcactgtgaatagcaaattcttcatgcatccaagacttaagcccagcatctttcaggcaggaaaacaatctacccctgaatggcatttttcttcttttctttttacctttggggcaggttctgtgttgacctttgaactaaccctataaccccagcaggtgttgaacctataatccccctgcctcatcctctcaaatagctggaatttgtgggccagcaccaagaggcccaaagaaagagaaagtttaaattaaatagaagaaaatgtagaggtattgataggtggtggtatcacacacctttaatcccagcactcaggaggcagtgacagattgctatctgtgagttcaaggttaggctggtctatagggctagttctagggtagccagagccacacaaagaaaacatgtctccaaaaagcaaacaaaaatgtagaggtgcacatttgggatctgggagcaggtaaaacttaaattcctagctagcttcaaatatataggaaaattctatcttacataaatcaattttttaaaaaaatctataaccgtatatcagaaaaacatagtaagatgaaaaatatatttgaaaaggaggtaccaaaatggtcctgagggactttggcttcttgatttattcattcaataaatgcaatacatttattcagtcaataagtcaatgtttattttgtattgaacgattctatgtactgatggtagagggaacagaatctttcttgaggtattaaaattctgggtgctttttgtcctcacaaggttaaagaaatttacttctgacatgtgggttttctttttaaaattataatcatgtaactttgtaactgggagaatacttggttgtgtttcttctaaattaatacttttaaaaatatttctttagtgttgggggtggctttacacagggaccggaccacatcacccacgtggaggtcagagatcaacttggaggagttgactctttccttgcaccaaatgggttctgcgggtcaaactcgggtcatcgggcttggtgttaccggctggcctctctcatctgccctagatctcagggggccctgctgacactctgctggtgtgtagattagaatcgcggcagctcggcgggtgcagaatgcccattgctccgttttgattcttctgcaacctgtatgtgactagcaacctcaccaagctgcagtctcttcctcggcaaaacagcataagaatgattctcagtctgaagttagtttaaagaggaaacaggaaacgaaagaacattaactagaatagcacttggcacacgacagacaactcatattagctagtttgctttccctatcttgggatatcatgattccaaaaatgagattttcccggagactgtatttcaaaaatagaactgctttgggttgtcagggaaataaatgatgccttcctgaaataataataataataacaacaacaacaacaacaacaatataaaacaaaaaccagccccatgctttcctagacagtgtaacctggtgacacatgaaatgtgtctcactttccccagaacttgaagccttgactcaggatgctcaacaggagggaagtaagagacaccaccccccacccccgcttgctgttttagcctcaccttgcaagtccccagtggacttaaatcttggaaaaggtgagcaccctgccctgaggtgcccaggccggaagcctagcacttcacatgaggttatgaaatcacacataattgtcaattgtctgaggagagattaatgtcatccagctggctttttcaaaagggtgtgaactggacggatgaataatgctcagagggaagaatggtagttcctctgatggggaggttctgttgactctgtcattcttccattcggtgggcggagccagttgttggataaacagatccaacctgattataaggggggaaaaagatgtgccctctgtcttttagggttacggccggtgga

SEQ ID NO: 2 - DNA, 20 bp, Artificial sequence
  Deleted region from Locus chr7_13872039
caggccagtttcatcccttc

SEQ ID NO: 3 - DNA, 19 bp, Artificial sequence
  Forward primer for deletion from chr7_13872039
attcccaaagcgtcgaact

SEQ ID NO: 4 - DNA, 22 bp, Artificial sequence
  Reverse primer for deletion from chr7_13872039
ctaccattggaaggacgatcac

SEQ ID NO: 5 - DNA, 13 bp, Artificial sequence
  LNA Probe for deletion from chr7_13872039 with locked nucleotides at n = 3,4,6,8,9,11
taaggacatccat

SEQ ID NO: 6 - DNA, 23 bp, Artificial sequence
  Forward primer for deletion from chr19_39237841
caattctttaggtgtaccctgtg

SEQ ID NO: 7 - DNA, 22 bp, Artificial sequence
  Reverse primer for deletion from chr19_39237841
aaacaatggagcagatgacatt

SEQ ID NO: 8 - DNA, 10 bp, Artificial sequence
  LNA Probe for deletion from chr19_39237841 with locked nucleotides at n = 2,3,4,6,8,9
ttctcgctgt